(19) United States
(12) Patent Application Publication
(10) Pub. No.: US 2024/0115582 A1
(43) Pub. Date: Apr. 11, 2024

(54) USE OF SODIUM TRANS-[TETRACHLORIDOBIS(1H-INDAZOLE)RUTHENATE(III)] FOR TREATING CANCERS

(71) Applicant: Bold Therapeutics Inc., Vancouver (CA)

(72) Inventors: Mark BAZETT, Vancouver, BC (CA); Adam CARIE, Vancouver, BC (CA); James PANKOVICH, Vancouver, BC (CA); Robbie CARSON, Belfast (GB); Sandra VAN SCHAEYBROECK, Belfast (GB); Paromita RAHA, Vancouver, BC (CA)

(21) Appl. No.: 18/253,296

(22) PCT Filed: Nov. 18, 2021
(86) PCT No.: PCT/CA2021/051639

§ 371 Date: May 17, 2023

Related U.S. Application Data
(60) Provisional application No. 63/115,587, filed on Nov. 18, 2020.
(60) Provisional application No. 63/165,641, filed on Mar. 24, 2021.

(51) Int. Cl. A61K 31/555; A61K 31/713; A61K 45/06; A61P 35/00; C12Q 1/6886

(52) U.S. Cl. CPC A61K 31/555; A61K 31/713; A61K 45/06; A61P 35/00; C12Q 1/6886; C12Q 2600/156; C12Y 207/11001

(57) ABSTRACT

Methods and corresponding uses are provided for treating a cancer in a patient in need thereof, comprising administering an effective amount of sodium trans-[tetrachloridobis(1H-indazole)ruthenate(III)] (BOLD-100). BOLD-100 may be used in combination with an effective amount of an inhibitor of ataxia-telangiectasia mutated and Rad3-related protein kinase (ATRi).

### FIELD

The invention is in the field of therapeutic compounds, including the combined use of sodium trans-[tetrachloridobis(1H-indazole)ruthenate(III)] and other therapeutics for treating cancers.

### BACKGROUND

Sodium trans-[tetrachloridobis(1H-indazole)ruthenate(III)] is a coordinated complex of ruthenium having anticancer activity (also known as BOLD-100, KP1339, NKP-1339, IT-139, and Na[RuIIICl4(Hind)2]). Methods of making alkali metal salts of trans-[tetrachlorobis(1H-indazole)ruthenate (III)] are for example described in PCT Patent Publication WO2018204930, such compounds having Formula I:

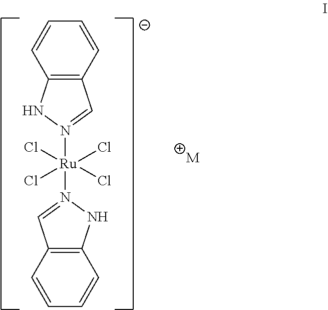

wherein M is an alkali metal cation, including the sodium salt:

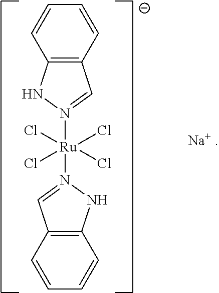

The MAPK (mitogen-activated protein kinase) pathway involves a RAS/RAF/MEK/ERK signaling cascade that is involved in cell proliferation, differentiation, survival and apoptosis. Within this cascade, mutations in RAS and RAF are common oncogenes in human cancer. Multiple signals activate the RAS family of GTPases (KRAS, NRAS and HRAS), which in turn activate downstream RAF protein kinases (ARAF, BRAF and CRAF). Within the RAF family of protein kinases, BRAF is a frequently mutated potent activator of MEK. BRAF mutations (BRAFMT) occur in ˜10-15% of metastatic colorectal cancer (mCRC) and correlate with a poor clinical outcome, in particular those with microsatellite stable (MSS) disease as distinct from disease characterized by microsatellite instability (MSI). For these reasons, the profiling of RAS (KRAS and NRAS) and BRAF genes and the assessment of mismatch repair (MMR)/MSI status may be useful as a diagnostic and therapeutic indicators in CRC.

ATR (Ataxia-Telangiectasia Mutated (ATM) and Rad3-related protein kinase) is a central component of the cellular DNA damage response (DDR). Replication protein A-coated single-strand DNA (ssDNA) at sites of DNA damage or stressed replication forks activates ATR, which then functions to activate a cell cycle checkpoint and suppress replication stress. Cancer cells are often characterized by features, such as replication stress, that invoke reliance on the ATR checkpoint function. Within this context, ATR inhibitors (ATRi) have shown promise as cancer therapeutics (WO2017118734, US20190365745, WO2016112374, WO2017180723, WO2018029117). Commercial ATRi include AZD6783, M4344 (formerly VX-803), VE-821, M6620 (formerly VX-970, berzosertib or VE-822), and BAY1895344 (Mei, L., Zhang, J., He, K. et al. Ataxia telangiectasia and Rad3-related inhibitors and cancer therapy: where we stand. J Hematol Oncol 12, 43 (2019)).

### SUMMARY

Methods and corresponding uses are provided for treating a cancer in patient in need thereof, such as a human patient, comprising administering an effective amount of sodium trans-[tetrachloridobis(1H-indazole)ruthenate(III)] (BOLD-100). The BOLD-100 may be used in combination with an effective amount of an inhibitor of ataxia-telangiectasia mutated and Rad3-related protein kinase (ATRi). The effective amount of BOLD-100 and the effective amount of the ATRi may for example be synergistically effective for treating the cancer. The cancer may be a cancer that is resistant to treatment with BOLD-100 alone, or is a cancer that is resistant to treatment with the ATRi alone, or a cancer that is resistant to another chemotherapy agent or chemotherapy regimen, and may for example be a metastatic cancer. The cancer may be a colorectal cancer (CRC), such as a CRC adenocarcinoma. The cancer may be characterized by a BRAF mutation (BRAFMT). The cancer may further be characterized by microsatellite stability (MSS). A select embodiment accordingly involves monotherapy with BOLD-100, or combination therapy using BOLD-100 with an ATRi, in the treatment of MSS BRAFMT CRC, such as metastatic MSS BRAFMT CRC. In combination therapies, BOLD-100 and the ATRi may be administered sequentially, in any order, or administered in combination, in a co-formulation or separately.

### BRIEF DESCRIPTION OF THE DRAWINGS

FIG. 1 includes two heatmaps, each with a consensus molecular subtype (CMS) profile along the X axis and, in the right hand heatmap, a Pathway name (Hallmark) along the Y axis, illustrating that the unfolded protein response (UPR) and DNA repair are dominant pathways deregulated in the CMS1/BRAFMT subgroup with the poorest outcome.

FIG. 2 includes an upper panel, with (A) a Western blot image and (B) three bar graphs; and a lower panel with two bar graphs (A and B), together illustrating that BRAFMT, MSS CRC cells are sensitive to treatment with BOLD-100.

FIG. 3 includes 3 panels, with (A) a line graph above a blot image, (B) a blot image, and (C) a blot image and a scatter plot, together illustrating that oncogenic BRAF is a determinant of response to BOLD-100 treatment.

FIG. 4 includes (A) two tables, (B) a scatter plot, (C) a blot image above a bar graph, (D) two blot images adjacent to two bar graphs, together illustrating that BOLD-100-induced cell death is dependent on caspase 8.

FIG. 5 includes (A) a heatmap and (B) two schematics, together illustrating that BOLD-100 treatment results in DNA damage repair pathway activation in BRAFMT CRC.

FIG. 6 includes three panels, with (A) two heatmaps and a bar graph, (B) two heatmaps and a bar graph, and (C) an image of cell cultures and three bar graphs, together illustrating that ATR inhibition markedly increases response to BOLD-100 treatment in BRAFMT CRC cells.

FIG. 7 includes three panels, with (A) two blot images, (B) two bar graphs, and (C) three bar graphs, together illustrating that the ATRi compounds AZD6738, M4344 and Berzosertib increase cell death following BOLD-100 treatment in BRAFMT CRC.

FIG. 8 includes four panels with (A) two blot images, (B) two blot images, (C) two bar graphs and (D) a blot image and a bar graph, together illustrating that BOLD-100 induces ROS-dependent ATR/CHK1 kinase activation and cell death in BRAFMT CRC cells.

FIG. 9 is a bar graph illustrating that BOLD-100 is most effective in treating challenging CMS1 and CMS4 CRC.

FIG. 10 includes two panels with (A) bar graphs over corresponding scatter plots, and (B) a scatter plot, illustrating that ATR inhibition markedly increases response to BOLD-100 treatment in multiple myeloma.

### DETAILED DESCRIPTION

As disclosed herein, BOLD-100 is shown to have a dramatic impact on the survival of BRAFMT CRC cells. Accordingly, therapies are provided for treating BRAFMT CRC with BOLD-100. Further, synergies are disclosed in the treatment of cancer cells, exemplified by BRAFMT CRC cells, with BOLD-100 in combination with inhibitors of ataxia-telangiectasia mutated and Rad3-related protein kinase (ATRi). Accordingly, therapies are provided for treating cancers, including BRAFMT CRC, with BOLD-100 in combination with an ATRi.

Using a panel of isogenic paired and non-isogenic V600E BRAFMT and BRAFWT cells, in vitro CellTitre-Glo® and Annexin V/PI sensitivity studies showed that BRAFMT, MSS CRC cells were highly sensitive to BOLD-100 with IC₅₀ values between 9.25-31 μM Treatment with BOLD-100 resulted in early decreases in GRP78 levels and increases in expression levels of the endoplasmic reticulum stress protein CHOP. This was associated with a caspase-8 dependent cell death in the BRAFMT CRC cells. Notably, silencing of CHOP did not abrogate BOLD-100-induced cell death in BRAFMT CRC cells, indicating that the unfolded protein response (UPR) pathway played no role in the cell death following BOLD-100. RNA seq and IPA bioinformatics analysis showed that cell cycle regulation and DNA repair were the most significant deregulated pathways following BOLD-100 treatment. Further mechanistic studies revealed that BOLD-100 induced rapid and potent increases in pATR^{T1989}, pChk1^{S345} and γH2AX expression levels in BRAFMT cells.

The ATRi compounds AZD6783 and M4344 resulted in strong synergy and apoptosis when combined with BOLD-100, in particular in BRAFMT CRC cells. Notably, the ROS scavenger NAC abrogated BOLD-100 induced CHOP, pATR^{T1989}, pChk1^{S345} and γH2AX levels and rescued cell death following BOLD-100 treatment in BRAFMT CRC cells.

Additional embodiments of the present invention provide methods for preparing drug products containing the sodium salt of trans-[tetrachlorobis(1H-indazole)ruthenate (III)] (i.e. BOLD-100).

One aspect of the current invention provides a method for preparing a sterile, lyophilized drug product containing sodium trans-[tetrachlorobis(1H-indazole)ruthenate (III)]. This formulation would be suitable for administration to a patient. The formulation is comprised of sodium trans-[tetrachlorobis(1H-indazole)ruthenate (III)], a pH buffer, and a cryoprotective agent. The general method for providing said formulation comprises the steps of preparing aqueous buffer solution, preparing aqueous cryoprotectant solution, dissolution of sodium trans-[tetrachlorobis(1H-indazole)ruthenate (III)] in the buffer solution, addition of the cryoprotectant solution, sterile filtration (e.g. aseptic filtration), filling of vials under sterile conditions, and lyophilization under sterile conditions. Suitable buffers include, but are not limited to: citrate, TRIS, acetate, EDTA, HEPES, tricine, and imidazole. The use of a phosphate buffer is possible but is not preferred. A preferred aspect of the present invention is the use of a citric acid/sodium citrate buffer. Suitable cryoprotective agents include, but are not limited to: sugars, monosaccarides, disaccharides, polyalcohols, mannitol, sorbitol, sucrose, trehalose, dextran, and dextrose. A preferred aspect of the present invention is the use of mannitol as the cryoprotective agent.

As described above, herein, sodium trans-[tetrachlorobis(1H-indazole)ruthenate (III)] can degrade in water to Compound A (Scheme II). One skilled in the art will recognize that limiting this degradation reaction would be advantageous to obtaining the highest purity product. It was found that cooling the sodium trans-[tetrachlorobis(1H-indazole)ruthenate (III)] solution during the formulation process was found to greatly reduce the amount of Compound A present in the lyophilized product. In one aspect of the invention, the sodium trans-[tetrachlorobis(1H-indazole)ruthenate (III)] solution is cooled to 4° C. during the formulation process. In another aspect of the invention, the sodium trans-[tetrachlorobis(1H-indazole)ruthenate (III)] solution is cooled to 2-8° C. during the formulation process. In another aspect of the invention, the sodium trans-[tetrachlorobis(1H-indazole)ruthenate (III)] solution is cooled to 2-15° C. during the formulation process.

One embodiment of the present invention provides a composition comprising sodium trans-[tetrachlorobis(1H-indazole)ruthenate (III)], a suitable buffer, and mannitol. In some embodiments, a suitable buffer comprises a citrate buffer. For instance, in some embodiments, a citrate buffer comprises sodium citrate and citric acid.

One embodiment of the present invention provides a composition comprising sodium trans-[tetrachlorobis(1H-indazole)ruthenate (III)], sodium citrate, citric acid, and mannitol.

One embodiment of the present invention provides a composition comprising sodium trans-[tetrachlorobis(1H-indazole)ruthenate (III)], sodium citrate, citric acid, mannitol, and mer,trans-[RuIIICl3(Hind)2(H2O)].

One embodiment of the present invention provides a composition comprising sodium trans-[tetrachlorobis(1H-indazole)ruthenate (III)], sodium citrate, citric acid, mannitol, mer,trans-[RuIIICl3(Hind)2(H2O)], and a cesium salt.

One embodiment of the present invention provides a composition comprising sodium trans-[tetrachlorobis(1H-indazole)ruthenate (III)], sodium citrate, citric acid, and mannitol, wherein the sodium trans-[tetrachlorobis(1H-indazole)ruthenate (III)] is amorphous.

One embodiment of the present invention provides a composition comprising sodium trans-[tetrachlorobis(1H-indazole)ruthenate (III)], sodium citrate, citric acid, mannitol, and mer,trans-[RuIIICl3(Hind)2(H2O)], wherein the sodium trans-[tetrachlorobis(1H-indazole)ruthenate (III)] is amorphous.

One embodiment of the present invention provides a composition comprising sodium trans-[tetrachlorobis(1H-indazole)ruthenate (III)], sodium citrate, citric acid, mannitol, mer,trans-[RuIIICl3(Hind)2(H2O)], and a cesium salt, wherein the sodium trans-[tetrachlorobis(1H-indazole)ruthenate (III)] is amorphous.

One embodiment of the present invention provides a composition comprising sodium trans-[tetrachlorobis(1H-indazole)ruthenate (III)], sodium citrate, citric acid, mannitol, mer,trans-[RuIIICl3(Hind)2(H2O)], and a cesium salt;

wherein:
mer,trans-[Ru^{III}Cl₃ (Hind)₂(H₂O)] is between about 0.01 and about 0.4 weight percent of the composition,
and cesium is between about 0.00001 and about 0.01 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III} Cl₃ (Hind)₂(H₂O)], and a cesium salt;

wherein:
mer,trans-[Ru^{III} Cl₃ (Hind)₂(H₂O)] is between about 0.01 and about 0.4 weight percent of the composition,
and cesium is between about 0.00001 and about 0.01 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III} Cl₃ (Hind)₂(H₂O)], and a cesium salt;

wherein:
mer,trans-[Ru^{III} Cl₃ (Hind)₂(H₂O)] is between about 0.01 and about 0.2 weight percent of the composition,
and cesium is between about 0.00001 and about 0.01 weight percent of the composition.

One embodiment of the present invention provides a composition comprising sodium trans-[tetrachlorobis(1H-indazole)ruthenate (III)], mer,trans-[Ru^{III}Cl₃ (Hind)₂(H₂O)], and a cesium salt;

wherein:
mer,trans-[Ru^{III}Cl₃ (Hind)₂(H₂O)] is between about 0.01 and about 0.40 weight percent of the composition,
and cesium is between about 0.00001 and about 0.01 weight percent of the composition.

One embodiment of the present invention provides a composition comprising sodium trans-[tetrachlorobis(1H-indazole)ruthenate (III)], mer,trans-[Ru^{III}Cl₃ (Hind)₂(H₂O)], and a cesium salt;

wherein:
the composition is a lyophilized powder,
mer,trans-[Ru^{III} Cl₃ (Hind)₂(H₂O)] is between about 0.01 and about 0.40 weight percent of the composition,
and cesium is between about 0.00001 and about 0.01 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III} Cl₃ (Hind)₂(H₂O)], and a cesium salt;

wherein:
the composition is a lyophilized powder,
mer,trans-[Ru^{III} Cl₃ (Hind)₂(H₂O)] is between about 0.01 and about 0.3 weight percent of the composition,
and cesium is between about 0.00001 and about 0.1 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III}Cl₃(Hind)₂(H₂O)], and a cesium salt;

wherein:
mer,trans-[Ru^{III}Cl₃(Hind)₂(H₂O)] is between about 0.01 and about 0.3 weight percent of the composition,
and cesium is between about 0.00001 and about 0.1 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III}Cl₃(Hind)₂(H₂O)], and a cesium salt;

wherein:
the composition is a lyophilized powder,
sodium trans-[tetrachlorobis(1H-indazole)ruthenate (III)] is about 11.5 to about 14.0 weight percent of the composition,
citric acid is about 43.9 to about 53.7 weight percent of the composition,
sodium citrate is about 25.7 to about 23.1 weight percent of the composition,
mannitol is about 11.5 to about 14.0 weight percent of the composition,
mer,trans-[Ru^{III}Cl₃(Hind)₂(H₂O)] is about 0.01 and about 0.3 weight percent of the composition,
and cesium is between about 0.00001 and about 0.1 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III}Cl₃(Hind)₂(H₂O)], and a cesium salt;

wherein:
the composition is a lyophilized powder,
sodium trans-[tetrachlorobis(1H-indazole)ruthenate (III)] is about 10.2 to about 15.3 weight percent of the composition,
citric acid is about 39.0 to about 58.5 weight percent of the composition,
sodium citrate is about 20.5 to about 30.8 weight percent of the composition,
mannitol is about 10.2 to about 15.3 weight percent of the composition,
mer,trans-[Ru^{III}Cl₃(Hind)₂(H₂O)] is about 0.01 and about 0.3 weight percent of the composition,
and cesium is between about 0.00001 and about 0.1 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III} Cl₃ (Hind)₂(H₂O)], and a cesium salt;

wherein:
the composition is a lyophilized powder,
sodium trans-[tetrachlorobis(1H-indazole)ruthenate (III)] is about 10.2 to about 15.3 weight percent of the composition,
mer,trans-[Ru^{III}Cl₃ (Hind)₂(H₂O)] is about 0.01 and about 0.3 weight percent composition,
and cesium is between about 0.00001 and about 0.1 weight percent of the composition.

One embodiment of the present invention provides a composition comprising sodium trans-[tetrachlorobis(1H-indazole)ruthenate (III)], mannitol, citric acid, and sodium citrate;

wherein:
sodium trans-[tetrachlorobis(1H-indazole)ruthenate (III)] is about 49.86 weight percent of the composition,
mannitol is about 49.86 weight percent of the composition,
citric acid is about 0.187 weight percent of the composition,
and sodium citrate is about 0.093 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, and sodium citrate;

wherein:
sodium trans-[tetrachlorobis(1H-indazole)ruthenate (III)] is about 40 to about 60 weight percent of the composition,
mannitol is about 40 to about 60 weight percent of the composition,
citric acid is about 0.01 to about 0.5 weight percent of the composition,
and sodium citrate is about 0.001 to about 0.25 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, and sodium citrate;

wherein:
sodium trans-[tetrachlorobis(1H-indazole)ruthenate (III)] is about 30 to about 70 weight percent of the composition,
mannitol is about 30 to about 70 weight percent of the composition,
citric acid is about 0.001 to about 1 weight percent of the composition,
and sodium citrate is about 0.0001 to about 1 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, and Ru^{III}Cl₃(Hind)₂(H₂O);

wherein:
sodium trans-[tetrachlorobis(1H-indazole)ruthenate (III)] is about 49.86 weight percent of the composition,
mannitol is about 49.86 weight percent of the composition,
citric acid is about 0.187 weight percent of the composition,
sodium citrate is about 0.093 weight percentage of the composition,
and Ru^{III}Cl₃(Hind)₂(H₂O) is not more than 0.5 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, and Ru^{III}Cl₃(Hind)₂(H₂O);

wherein:
sodium trans-[tetrachlorobis(1H-indazole)ruthenate (III)] is about 40 to about 60 weight percent of the composition,
mannitol is about 40 to about 60 weight percent of the composition,
citric acid is about 0.01 to about 0.5 weight percent of the composition,
sodium citrate is about 0.001 to about 0.25 weight percentage of the composition,
and Ru^{III}Cl₃(Hind)₂(H₂O) is about 0 to about 0.5 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), and cesium;

wherein:
sodium trans-[tetrachlorobis(1H-indazole)ruthenate (III)] is about 30 to about 70 weight percent of the composition,
mannitol is about 30 to about 70 weight percent of the composition,
citric acid is about 0.001 to about 1 weight percent of the composition,
sodium citrate is about 0.0001 to about 1 weight percentage of the composition,
Ru^{III}Cl₃(Hind)₂(H₂O) is not more than 0.5 weight percentage of the composition,
and cesium is not more than 0.25 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, and cesium;

wherein:
sodium trans-[tetrachlorobis(1H-indazole)ruthenate (III)] is about 49.61 weight percent of the composition,
mannitol is about 49.86 weight percent of the composition,
citric acid is about 0.187 weight percent of the composition,
sodium citrate is about 0.093 weight percentage of the composition
and cesium is about 0.25 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, and cesium;

wherein:
sodium trans-[tetrachlorobis(1H-indazole)ruthenate (III)] is about 40 to about 60 weight percent of the composition,
mannitol is about 40 to about 60 weight percent of the composition,
citric acid is about 0.01 to about 0.5 weight percent of the composition,
sodium citrate is about 0.001 to about 0.25 weight percentage of the composition,
and cesium is about 0.1 to about 0.5 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, and cesium;

wherein:
sodium trans-[tetrachlorobis(1H-indazole)ruthenate (III)] is about 30 to about 70 weight percent of the composition,
mannitol is about 30 to about 70 weight percent of the composition,
citric acid is about 0.001 to about 1 weight percent of the composition,
sodium citrate is about 0.0001 to about 1 weight percentage of the composition,
and cesium is about 0.01 to about 1 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium;

wherein:
sodium trans-[tetrachlorobis(1H-indazole)ruthenate (III)] about 46.61 weight percent of the composition,
mannitol is about 49.86 weight percent of the composition,
citric acid is about 0.187 weight percent of the composition,
sodium citrate is about 0.093 weight percentage of the composition,
Ru^{III}Cl₃(Hind)₂(H₂O) is not more than 0.5 weight percentage of the composition,
Ru^{III}Cl₃(Hind)₂(CH₃CN) is not more than 1.25 weight percentage of the composition,
Ru^{III}Cl₃(Hind)(HN═C(Me)ind) is not more than 1.0 weight percentage of the composition,
and cesium is not more than 0.25 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium;

wherein:
sodium trans-[tetrachlorobis(1H-indazole)ruthenate (III)] about between 46.61 weight percent of the composition,
mannitol is about 49.86 weight percent of the composition,
citric acid is about 0.187 weight percent of the composition,
sodium citrate is about 0.093 weight percentage of the composition,
Ru^{III}Cl₃(Hind)₂(H₂O) is not more than 0.5 weight percentage of the composition,
Ru^{III}Cl₃(Hind)₂(CH₃CN) is not more than 1.25 weight percentage of the composition,
Ru^{III}Cl₃(Hind)(HN═C(Me)ind) is not more than 1.0 weight percentage of the composition,
and cesium is not more than 0.25 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium;

wherein:
sodium trans-[tetrachlorobis(1H-indazole)ruthenate (III)] is about 40 to about 60 weight percent of the composition,
mannitol is about 40 to about 60 weight percent of the composition,
citric acid is about 0.01 to about 0.5 weight percent of the composition,
sodium citrate is about 0.001 to about 0.25 weight percentage of the composition,
Ru^{III}Cl₃(Hind)₂(H₂O) is not more than about 0.5 weight percentage of the composition,
Ru^{III}Cl₃(Hind)₂(CH₃CN) is not more than about 1.25 weight percentage of the composition,
Ru^{III}Cl₃(Hind)(HN═C(Me)ind) is not more than about 1.0 weight percentage of the composition,
and cesium is not more than 0.25 percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium;

wherein:
sodium trans-[tetrachlorobis(1H-indazole)ruthenate (III)] is about 30 to about 70 weight percent of the composition,
mannitol is about 30 to about 70 weight percent of the composition,
citric acid is about 0.001 to about 1 weight percent of the composition,
sodium citrate is about 0.0001 to about 1 weight percentage of the composition,
Ru^{III}Cl₃(Hind)₂(H₂O) is not more than about 0.5 weight percentage of the composition,
Ru^{III}Cl₃(Hind)₂(CH₃CN) is not more than about 1.25 weight percentage of the composition,
Ru^{III}Cl₃(Hind)(HN═C(Me)ind) is not more than about 1.0 weight percentage of the composition,
and cesium is not more than 0.25 percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium;

wherein:
sodium trans-[tetrachlorobis(1H-indazole)ruthenate (III)] is about 20 to about 80 weight percent of the composition,
mannitol is about 20 to about 80 weight percent of the composition,
citric acid is about 0.0001 to about 5 weight percent of the composition,
sodium citrate is about 0.00001 to about 5 weight percentage of the composition,
Ru^{III}Cl₃(Hind)₂(H₂O) is not more than about 0.5 weight percentage of the composition,
Ru^{III}Cl₃(Hind)₂(CH₃CN) is not more than about 1.25 weight percentage of the composition,
Ru^{III}Cl₃(Hind)(HN═C(Me)ind) is not more than about 1.0 weight percentage of the composition,
and cesium is not more than 0.25 percentage of the composition. In some such embodiments, the composition is a lyophilized powder.

In some embodiments, the present invention provides a unit dosage form comprising a formulation or composition described herein. The expression “unit dosage form” as used herein refers to a physically discrete unit of a provided formulation appropriate for the subject to be treated. It will be understood, however, that the total daily usage of provided formulation will be decided by the attending physician within the scope of sound medical judgment. The specific effective dose level for any particular subject or organism will depend upon a variety of factors including the disorder being treated and the severity of the disorder; activity of specific active agent employed; specific formulation employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

Compositions of the present invention can be provided as a unit dosage form. In some embodiments, a vial comprising sodium trans-[tetrachlorobis(1H-indazole)ruthenate (III)], mannitol, citric acid, sodium citrate is a unit dosage form.

In some embodiments, the present invention a vial comprising sodium trans-[tetrachlorobis(1H-indazole)ruthenate (III)], mannitol, citric acid, sodium citrate, and cesium is a unit dosage form.

In some embodiments, the present invention a vial comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium is a unit dosage form.

Still further encompassed by the invention are pharmaceutical packs and/or kits comprising compositions described herein, or a unit dosage form comprising a provided composition, and a container (e.g., a foil or plastic package, or other suitable container). Optionally instructions for use are additionally provided in such kits.

In some embodiments, the present invention can be provided as a unit dosage form. Indeed, a vial comprising sodium trans-[tetrachlorobis(1H-indazole)ruthenate (III)], mannitol, citric acid, sodium citrate is a unit dosage form depicted in Table 3

TABLE 3
Pharmaceutical Components

| | | Weight | Amount/ | |
|---|---|---|---|---|
| Component | Function | % | vial | |
| sodium trans-[tetrachlorobis(1H- | Active | 47.5 | 100 | mg |
| indazole)ruthenate (III)] | | | | |
| Mannitol | Cryoprotectant | 47.5 | 100 | mg |
| Citric Acid | Buffer component | 3.37 | 7.1 | mg |
| Sodium citrate | Buffer component | 1.63 | 3.4 | mg |

In some embodiments, the pharmaceutical components described in Table 3 further comprise cesium;

wherein:

cesium is not more than 0.25 weight percentage of the composition.

In some embodiments, the pharmaceutical components described in Table 3 further comprise cesium, Ru^{III}Cl₃(Hind)₂(H₂O), Ru^{III}Cl₃(Hind)₂(CH₃CN), and Ru^{III}Cl₃(Hind)(HN═C(Me)ind);

wherein:
cesium is not more than about 0.25 weight percentage of the composition,
Ru^{III}Cl₃(Hind)₂(H₂O) is not more than about 0.5 weight percentage of the composition,
Ru^{III}Cl₃(Hind)₂(CH₃CN) is not more than about 1.25 weight percentage of the composition,
and Ru^{III}Cl₃(Hind)(HN═C(Me)ind) is not more than about 1.0 weight percentage of the composition.

In some embodiments, the pharmaceutical composition is selected from those in Table 4:

TABLE 4
Pharmaceutical Component Ranges

| | | Weight % | | |
|---|---|---|---|---|
| Component | Function | Range | Amount/vial | |
| sodium trans-[tetrachlorobis(1H- | Active | 42.75-52.25 | 90-110 | mg |
| indazole)ruthenate (III)] | | | | |
| Mannitol | Cryoprotectant | 42.75-52.25 | 90-110 | mg |
| Citric Acid | Buffer component | 3.033-3.707 | 6.39-7.81 | mg |
| Sodium citrate | Buffer component | 1.467-1.793 | 3.06-3.74 | mg |

In some embodiments, the pharmaceutical components described in Table 4 further comprise cesium;

wherein:

cesium is not more than 0.25 weight percentage of the composition.

In some embodiments, the pharmaceutical components described in Table 4 further comprise cesium, Ru^{III}Cl₃(Hind)₂(H₂O), Ru^{III}Cl₃(Hind)₂(CH₃CN), and Ru^{III}Cl₃(Hind)(HN═C(Me)ind);

wherein:
cesium is not more than about 0.25 weight percentage of the composition,
Ru^{III}Cl₃(Hind)₂(H₂O) is not more than about 0.5 weight percentage of the composition,
Ru^{III}Cl₃(Hind)₂(CH₃CN) is not more than about 1.25 weight percentage of the composition,
and Ru^{III}Cl₃(Hind)(HN═C(Me)ind) is not more than about 1.0 weight percentage of the composition.

In some embodiments, the present invention can be provided as a unit dosage form. Indeed, a vial comprising sodium trans-[tetrachlorobis(1H-indazole)ruthenate (III)], mannitol, citric acid, sodium citrate is a unit dosage form depicted in Table 5:

TABLE 5
Pharmaceutical Components

| | | Weight | Amount/ | |
|---|---|---|---|---|
| Component | Function | % | vial | |
| sodium trans-[tetrachlorobis(1H- | Active | 49.86 | 300 | mg |
| indazole)ruthenate (III)] | | | | |
| Mannitol | Cryoprotectant | 49.86 | 300 | mg |
| Citric Acid | Buffer component | 0.188 | 1.13 | mg |
| Sodium citrate | Buffer component | 0.092 | 0.55 | mg |

In some embodiments, the pharmaceutical components described in Table 5 further comprise cesium;

wherein:

cesium is not more than 0.25 weight percentage of the composition.

In some embodiments, the pharmaceutical components described in Table 5 further comprise cesium, Ru^{III}Cl₃(Hind)₂(H₂O), Ru^{III}Cl₃(Hind)₂(CH₃CN), and Ru^{III}Cl₃(Hind)(HN═C(Me)ind);

wherein:
cesium is not more than about 0.25 weight percentage of the composition,
Ru^{III}Cl₃(Hind)₂(H₂O) is not more than about 0.5 weight percentage of the composition,
Ru^{III}Cl₃(Hind)₂(CH₃CN) is not more than about 1.25 weight percentage of the composition,
and Ru^{III}Cl₃(Hind)(HN═C(Me)ind) is not more than about 1.0 weight percentage of the composition.

In some embodiments, the pharmaceutical composition is selected from those in Table 6:

TABLE 6
Pharmaceutical Components

| | | Weight % | | |
|---|---|---|---|---|
| Component | Function | Range | Amount/vial | |
| sodium trans-[tetrachlorobis(1H- | Active | 44.87-54.85 | 270-330 | mg |
| indazole)ruthenate (III)] | | | | |
| Mannitol | Cryoprotectant | 44.87-54.85 | 270-330 | mg |
| Citric Acid | Buffer component | 0.169-0.207 | 1.02-1.24 | mg |
| Sodium citrate | Buffer component | 0.0828-0.1012 | 0.495-0.605 | mg |

In some embodiments, the pharmaceutical components described in Table 6 further comprise cesium;

wherein:

cesium is not more than 0.25 weight percentage of the composition.

In some embodiments, the pharmaceutical components described in Table 6 further comprise cesium, Ru^{III}Cl₃(Hind)₂(H₂O), Ru^{III}Cl₃(Hind)₂(CH₃CN), and Ru^{III}Cl₃(Hind)(HN═C(Me)ind);

wherein:
cesium is not more than about 0.25 weight percentage of the composition,
Ru^{III}Cl₃(Hind)₂(H₂O) is not more than about 0.5 weight percentage of the composition,
Ru^{III}Cl₃(Hind)₂(CH₃CN) is not more than about 1.25 weight percentage of the composition,
and Ru^{III}Cl₃(Hind)(HN═C(Me)ind) is not more than about 1.0 weight percentage of the composition.

In some embodiments, the pharmaceutical components are as described in any of Tables 3-6, and further comprise cesium. In some embodiments, cesium is present in an amount of about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, or 1.0 weight percentage of the composition.

In some embodiments, the present invention provides a method for treating cancer in a subject in need thereof comprising administering to the subject an effective amount of BOLD-100. In some embodiments, the subject is a human patient. In some embodiments, the cancer is a colorectal cancer (CRC), and may be characterized by a BRAF mutation (BRAFMT), and may be further characterized by microsatellite stability (MSS). In some embodiments, BOLD-100 may be used in in combination with a chemotherapeutic agent, such as an immuno-oncology agent, for example an inhibitor of ataxia-telangiectasia mutated and Rad3-related protein kinase (ATRi).

In some embodiments, the effective amount of BOLD-100 may be effective to reduce the amount of GRP78 in cancer cells following administration of BOLD-100, either alone or in combination, for example in combination with an ATRi.

According to one embodiment of the present invention provides a method for treating cancer in a patient in need thereof, comprising the steps of:
1) administering to the patient a chemotherapy agent (such as an ATRi);
2) subsequently administering BOLD-100, or a pharmaceutically acceptable composition thereof; to the patient; and
3) optionally repeating steps 1 and 2.

In certain embodiments, the BOLD-100, or a pharmaceutically acceptable composition thereof, is administered 1 day after the chemotherapy agent (such as an ATRi). In other embodiments, BOLD-100, or a pharmaceutically acceptable composition thereof, is administered to the patient 1 week after the chemotherapy agent (such as an ATRi). In yet other embodiments, BOLD-100 is administered to a patient between 1 and seven days after the chemotherapy agent (such as an ATRi).

In certain embodiments, the BOLD-100, or a pharmaceutically acceptable composition thereof, is administered simultaneously with the chemotherapy agent (such as an ATRi). In certain embodiments, the BOLD-100, or a pharmaceutically acceptable composition thereof, and the chemotherapy agent (such as an ATRi) are administered within about 20-28 hours of each other, or within about 22-26 hours of each other, or within about 24 hours of each other.

In certain embodiments, the BOLD-100, or a pharmaceutically acceptable composition thereof, is administered before the chemotherapy agent (such as an ATRi). In certain embodiments, the BOLD-100, or a pharmaceutically acceptable composition thereof, is administered at least about 8-16 hours before the chemotherapy agent (such as an ATRi), or at least about 10-14 hours before the chemotherapy agent (such as an ATRi), or at least about 12 hours before the chemotherapy agent (such as an ATRi).

In certain embodiments, the BOLD-100, or a pharmaceutically acceptable composition thereof, is administered at least about 20-28 hours before the chemotherapy agent (such as an ATRi), or at least about 22-26 hours before the chemotherapy agent (such as an ATRi), or at least about 24 hours before the chemotherapy agent (such as an ATRi).

In certain embodiments, the BOLD-100, or a pharmaceutically acceptable composition thereof, is administered at least about 44-52 hours before the chemotherapy agent (such as an ATRi), or at least about 46-50 hours before the chemotherapy agent (such as an ATRi), or at least about 48 hours before the chemotherapy agent (such as an ATRi).

In certain embodiments, the BOLD-100, or a pharmaceutically acceptable composition thereof, is administered at least about 64-80 hours before the chemotherapy agent (such as an ATRi), or at least about 70-74 hours before the chemotherapy agent (such as an ATRi), or at least about 72 hours before the chemotherapy agent (such as an ATRi).

In certain embodiments, the BOLD-100, or a pharmaceutically acceptable composition thereof, is administered before the chemotherapy agent (such as an ATRi). In certain embodiments, the BOLD-100, or a pharmaceutically acceptable composition thereof, is administered at least about 8-16 hours after the chemotherapy agent (such as an ATRi), or at least about 10-14 hours after the chemotherapy agent (such as an ATRi), or at least about 12 hours after the chemotherapy agent (such as an ATRi).

In certain embodiments, the BOLD-100, or a pharmaceutically acceptable composition thereof, is administered at least about 20-28 hours after the chemotherapy agent (such as an ATRi), or at least about 22-26 hours after the chemotherapy agent (such as an ATRi), or at least about 24 hours after the chemotherapy agent (such as an ATRi).

In certain embodiments, the BOLD-100, or a pharmaceutically acceptable composition thereof, is administered at least about 44-52 hours after the chemotherapy agent (such as an ATRi), or at least about 46-50 hours after the chemotherapy agent (such as an ATRi), or at least about 48 hours after the chemotherapy agent (such as an ATRi).

In certain embodiments, the BOLD-100, or a pharmaceutically acceptable composition thereof, is administered at least about 64-80 hours after the chemotherapy agent (such as an ATRi), or at least about 70-74 hours after the chemotherapy agent (such as an ATRi), or at least about 72 hours after the chemotherapy agent (such as an ATRi).

In certain embodiments, including in BRAFMT CRC patients (such as patients with metastatic disease), the chemotherapeutic agent regimen may involve use of: 5 fluorouracil, leucovorin and oxaliplatin (FOLFOX); or, 5-fluorouracil, leucovorin and irinotecan (FOLFIRI); or capecitabine plus oxaliplatin; or 5 fluorouracil, leucovorin, oxaliplatin and irinotecan with bevacizumab (FOLFOXIRI+bev).

In certain embodiments, the BOLD-100, or a pharmaceutically acceptable composition thereof, is administered simultaneously with an ATRi. In certain embodiments, where BOLD-100, or a pharmaceutically acceptable composition thereof, and ATRi are administered in combination, the two therapeutics may be administered within about 20-28 hours of each other, or within about 22-26 hours of each other, or within about 24 hours of each other.

In certain embodiments, the BOLD-100, or a pharmaceutically acceptable composition thereof, is administered before an ATRi. In certain embodiments, the BOLD-100, or a pharmaceutically acceptable composition thereof, is administered at least about 8-16 hours before an ATRi, or at least about 10-14 hours before an ATRi, or at least about 12 hours before an ATRi. In certain embodiments, the BOLD-100, or a pharmaceutically acceptable composition thereof, is administered at least about 20-28 hours before an ATRi, or at least about 22-26 hours before an ATRi, or at least about 24 hours before an ATRi. In certain embodiments, the BOLD-100, or a pharmaceutically acceptable composition thereof, is administered at least about 44-52 hours before an ATRi, or at least about 46-50 hours before an ATRi, or at least about 48 hours before an ATRi.

A titratable dosage may for example be adapted to allow a patient to take the medication in doses smaller than the unit dose, wherein a “unit dose” is defined as the maximum dose of medication that can be taken at any one time or within a specific dosage period. Titration of doses will allow different patients to incrementally increase the dose until they feel that the medication is efficacious, as not all patients will require the same dose to achieve the same benefits. A person with a larger build or faster metabolism may require larger doses to achieve the same effect as another with a smaller build or slower metabolism. Therefore, a titratable dosage has advantages over a standard dosage form.

In select embodiments, formulations may be adapted to be delivered in such a way as to target one or more of the following: sublingual, buccal, oral, rectal, nasal, parenteral and via the pulmonary system. Formulations may for example be in one or more of the following forms: gel, gel spray, tablet, liquid, capsule, by injection, or for vaporization.

Conventional pharmaceutical practice may be employed to provide suitable formulations or compositions to administer the formulations to subjects. Routes of administration may for example include, parenteral, intravenous, intradermal, subcutaneous, intramuscular, intracranial, intraorbital, ophthalmic, intraventricular, intracapsular, intraspinal, intrathecal, intracisternal, intraperitoneal, intranasal, inhalational, aerosol, topical, sublingual or oral administration. Therapeutic formulations may be in the form of liquid solutions or suspensions; for oral administration, formulations may be in the form of tablets or capsules; for intranasal formulations, in the form of powders, nasal drops, or aerosols; and for sublingual formulations, in the form of drops, aerosols or tablets.

Methods well known in the art for making formulations are found in, for example, “Remington: The Science and Practice of Pharmacy” (21st edition), ed. David Troy, 2006, Lippincott Williams & Wilkins. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Pharmaceutical compositions of the present invention may be in any form which allows for the composition to be administered to a patient. For example, the composition may be in the form of a solid, liquid or gas (aerosol). Pharmaceutical composition of the invention are formulated so as to allow the active ingredients contained therein to be bioavailable upon administration of the composition to a patient. Compositions that will be administered to a patient may take the form of one or more dosage units, where for example, a tablet, capsule or cachet may be a single dosage unit, and a container of the compound in aerosol form may hold a plurality of dosage units.

Materials used in preparing the pharmaceutical compositions should be pharmaceutically pure and non-toxic in the amounts used. The inventive compositions may include one or more compounds (active ingredients) known for a particularly desirable effect. It will be evident to those of ordinary skill in the art that the optimal dosage of the active ingredient(s) in the pharmaceutical composition will depend on a variety of factors. Relevant factors include, without limitation, the type of subject (e.g., human), the particular form of the active ingredient, the manner of administration and the composition employed.

In general, the pharmaceutical composition includes a formulation of the present invention as described herein, in admixture with one or more carriers. The carrier(s) may be particulate, so that the compositions are, for example, in tablet or powder form. The carrier(s) may be liquid, with the compositions being, for example, an oral syrup or injectable liquid. In addition, the carrier(s) may be gaseous, so as to provide an aerosol composition useful in, e.g., inhalatory administration.

When intended for oral administration, the composition is preferably in either solid or liquid form, where semi-solid, semi-liquid, suspension and gel forms are included within the forms considered herein as either solid or liquid.

As a solid formulation for oral administration, the composition may be formulated into a powder, granule, compressed tablet, pill, capsule, cachet, chewing gum, wafer, lozenges, or the like form. Such a solid composition will typically contain one or more inert diluents or edible carriers. In addition, one or more of the following adjuvants may be present: binders such as syrups, acacia, sorbitol, polyvinylpyrrolidone, carboxymethylcellulose, ethyl cellulose, microcrystalline cellulose, gum tragacanth or gelatin, and mixtures thereof; excipients such as starch, lactose or dextrins, disintegrating agents such as alginic acid, sodium alginate, Primogel, corn starch and the like; lubricants such as magnesium stearate or Sterotex; fillers such as lactose, mannitols, starch, calcium phosphate, sorbitol, methylcellulose, and mixtures thereof; lubricants such as magnesium stearate, high molecular weight polymers such as polyethylene glycol, high molecular weight fatty acids such as stearic acid, silica, wetting agents such as sodium lauryl sulfate, glidants such as colloidal silicon dioxide; sweetening agents such as sucrose or saccharin, a flavoring agent such as peppermint, methyl salicylate or orange flavoring, and a coloring agent. When the composition is in the form of a capsule, e.g., a gelatin capsule, it may contain, in addition to materials of the above type, a liquid carrier such as polyethylene glycol or a fatty oil.

The formulation may be in the form of a liquid, e.g., an elixir, syrup, solution, aqueous or oily emulsion or suspension, or even dry powders which may be reconstituted with water and/or other liquid media prior to use. The liquid may be for oral administration or for delivery by injection, as two examples. When intended for oral administration, preferred compositions contain, in addition to the present compounds, one or more of a sweetening agent, thickening agent, preservative (e.g., alkyl p-hydoxybenzoate), dye/colorant and flavor enhancer (flavorant). In a composition intended to be administered by injection, one or more of a surfactant, preservative (e.g., alkyl p-hydroxybenzoate), wetting agent, dispersing agent, suspending agent (e.g., sorbitol, glucose, or other sugar syrups), buffer, stabilizer and isotonic agent may be included. The emulsifying agent may be selected from lecithin or sorbitol monooleate.

The liquid pharmaceutical formulations of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

The pharmaceutical formulation may be intended for topical administration, in which case the carrier may suitably comprise a solution, emulsion, ointment, cream or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The formulation may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol. Low-melting waxes are preferred for the preparation of a suppository, where mixtures of fatty acid glycerides and/or cocoa butter are suitable waxes. The waxes may be melted, and the aminocyclohexyl ether compound is dispersed homogeneously therein by stirring. The molten homogeneous mixture is then poured into convenient sized molds, allowed to cool and thereby solidify.

The formulation may include various materials which modify the physical form of a solid or liquid dosage unit. For example, the composition may include materials that form a coating shell around the active ingredients. The materials which form the coating shell are typically inert, and may be selected from, for example, sugar, shellac, and other enteric coating agents. Alternatively, the active ingredients may be encased in a gelatin capsule or cachet.

The pharmaceutical formulation may consist of gaseous dosage units, e.g., it may be in the form of an aerosol. The term aerosol is used to denote a variety of systems ranging from those of colloidal nature to systems consisting of pressurized packages. Delivery may be by a liquefied or compressed gas or by a suitable pump system which dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit.

Some biologically active compounds may be in the form of the free base or in the form of a pharmaceutically acceptable salt such as the hydrochloride, sulfate, phosphate, citrate, fumarate, methanesulfonate, acetate, tartrate, maleate, lactate, mandelate, salicylate, succinate and other salts known in the art. The appropriate salt would be chosen to enhance bioavailability or stability of the compound for the appropriate mode of employment (e.g., oral or parenteral routes of administration).

The present invention also provides kits that contain a pharmaceutical formulation, together with instructions for the use of the formulation. Preferably, a commercial package will contain one or more unit doses of the formulation. Formulations which are light and/or air sensitive may require special packaging and/or formulation. For example, packaging may be used which is opaque to light, and/or sealed from contact with ambient air, and/or formulated with suitable coatings or excipients.

The formulations of the invention can be provided alone or in combination with other compounds (for example, small molecules, nucleic acid molecules, peptides, or peptide analogues), in the presence of a carrier or any pharmaceutically or biologically acceptable carrier. As used herein “pharmaceutically acceptable carrier” or “excipient” includes any and all solvents, dispersion media, coatings, antibacterial and antifungal agents, isotonic and absorption delaying agents, and the like that are physiologically compatible. The carrier can be suitable for any appropriate form of administration. Pharmaceutically acceptable carriers generally include sterile aqueous solutions or dispersions and sterile powders. Supplementary active compounds can also be incorporated into the formulations.

An “effective amount” of a formulation according to the invention includes a therapeutically effective amount or a prophylactically effective amount. A “therapeutically effective amount” refers to an amount effective, at dosages and for periods of time necessary, to achieve the desired therapeutic result. A therapeutically effective amount of a formulation may vary according to factors such as the disease state, age, sex, and weight of the individual, and the ability of the compound to elicit a desired response in the individual. Dosage regimens may be adjusted to provide the optimum therapeutic response. A therapeutically effective amount may also be one in which any toxic or detrimental effects of the formulation or active compound are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. For any particular subject, the timing and dose of treatments may be adjusted over time (e.g., timing may be daily, every other day, weekly, monthly) according to the individual need and the professional judgment of the person administering or supervising the administration of the compositions.

In therapeutic applications, synergy between active ingredients occurs when an observed combined therapeutic effect is greater than the sum of therapeutic effects of individual active ingredients, or a new therapeutic effect is produced that the active ingredients could not produce alone. Accordingly, when components of a formulation are present in synergistically effective amounts, the formulation yields a therapeutic effect that is greater than would be achieved by the individual active ingredients administered alone at comparable dosages. In this context, the enhancement of therapeutic effect may take the form of increased efficacy or potency and/or decreased adverse effects. The synergistic effect may be mediated in whole or in part by the pharmacokinetics and/or pharmacodynamics of the active ingredients in a subject, so that the amount and proportion of the ingredients in the formulation may be synergistic in vivo. This in vivo synergy may be effected with a formulation that includes the active ingredients in amounts and proportions that are also synergistic in in vitro assays of efficacy. As used herein, the term “synergistically effective amounts” accordingly refers to amounts that are synergistic in vivo and/or in vitro. A numeric quantification of synergy is often expressed as a fractional inhibitory concentration index (FICI), which represents the sum of the fractional inhibitory concentrations (FICs) of each drug tested, where the FIC is determined for each drug by dividing the minimum inhibitory concentration (MIC, the lowest concentration of the drug which prevents visible growth of the bacterium in a standard in vitro assay—standard colorometric assay based on resazurin) of each drug when used in combination by the MIC of each drug when used alone. In very general terms, a FICI lower or higher than 1 indicates positively correlated activity (at least additive synergy) or an absence of positive interactions, respectively. More definitively, synergy of two compounds may be conservatively defined as a FICI of ≤0.5 (see Odds, 2003; with additivity or additive synergy corresponding to a FICI of >0.5 to 1; no interaction (indifference) corresponding to a FICI of >1 to ≤4; and antagonism corresponding to a FICI of >>4). Synergy of three compounds has been defined as a FICI of ≤1.0. (Berenbaum, 1978; Yu et al., 1980).

### EXAMPLES

As illustrated in the following Examples, BRAFMT MSS cell lines show increased sensitivity to BOLD-100, an inducer of DNA replication stress and UPR activation. BOLD-100 results in acute increases in ROS and activation of the ATR/CHK DNA damage signalling kinases in BRAFMT CRC cells. ATR inhibition significantly decreases cell viability and increased cell death when combined with BOLD-100 in BRAFMT CRC.

### Example 1: Unfolded Protein Response (UPR) and DNA Repair are Dominant Pathways Deregulated in the CMS1/BRAFMT Subgroup with the Poorest Outcome

Data analysis of the GSE59857 dataset: Heatmaps were generated through the use of the CMScaller application using R Studio version 1.3.959. The CMScaller application provides Consensus Molecular Subtype (CMS) classification of CRC pre-clinical models (see Guinney et al., Nat Med. 2015 November; 21(11):1350-6; CMS1 “microsatellite instability immune”, hypermutated, microsatellite unstable and strong immune activation; CMS2 “canonical”, epithelial, marked WNT and MYC signaling activation; CMS3 “metabolic”, epithelial and evident metabolic dysregulation; and CMS4 “mesenchymal”, prominent transforming growth factor-13 activation, stromal invasion and angiogenesis).

Materials: A panel of isogenic paired and non-isogenic V600E BRAFMT and BRAFWT cells were used. BOLD-100, a ruthenium-based small molecule inhibitor, was obtained from BOLD Therapeutics. A FDA approved compound library was used.

Methods: Cell Titre Glo, Annexin V/PI high content screening, Flow Cytometry, Western blotting, Caspase 8, 3/7 activity, ROS-Glo^{a} H2O2, RNAi assays were used. RNA seq and bioinformatic analyses were performed on BOLD-100-treated BRAFMT/WT CRC cells, using the Illumina Novoseq platform and Reactome Pathway Analysis.

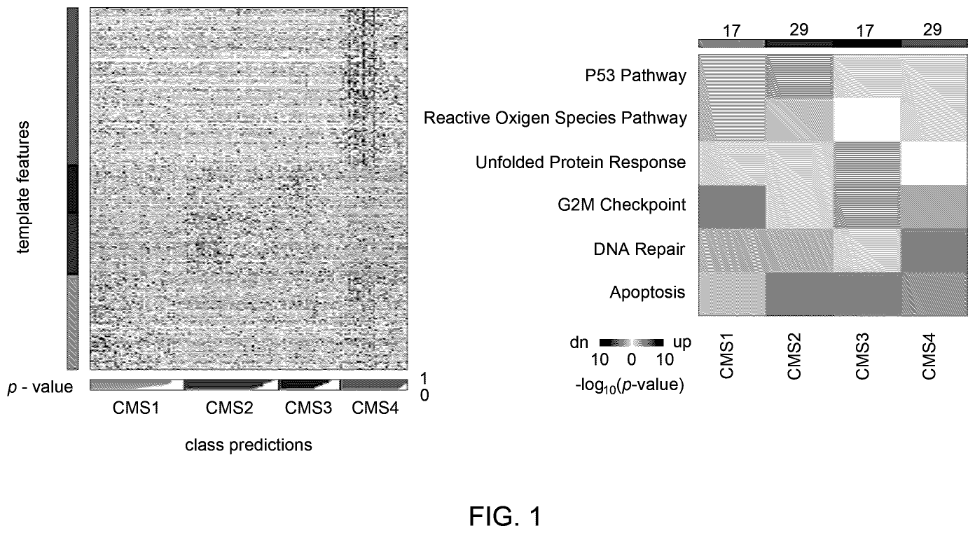

As Illustrated in FIG. 1, GSE59857 dataset was embedded into the platform upon its creation and as such, heatmaps were generated from this dataset which was Log 2 normalised, with the use of “Biobase” and “limma” packages in R for implementation of CMScaller. The “SubCamera” function within the CMScaller program is utilised to visualise heatmaps generated post gene set analysis and stratification by CMS profile. These heatmaps then contain a CMS profile along the X axis and Pathway name as per the Hallmark along the Y axis.

### Example 2: BRAFMT, MSS CRC Cells are Sensitive to Treatment with BOLD-100

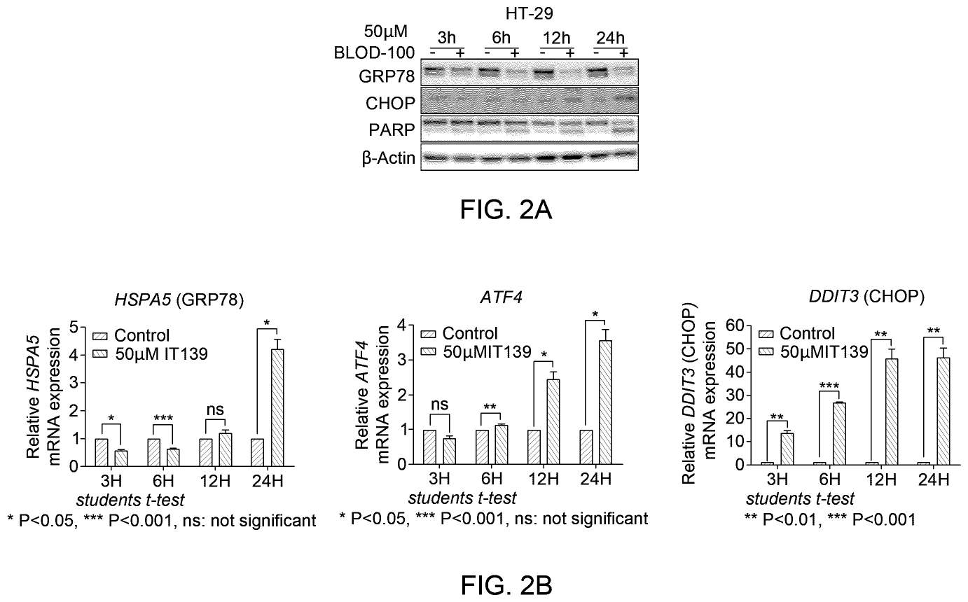

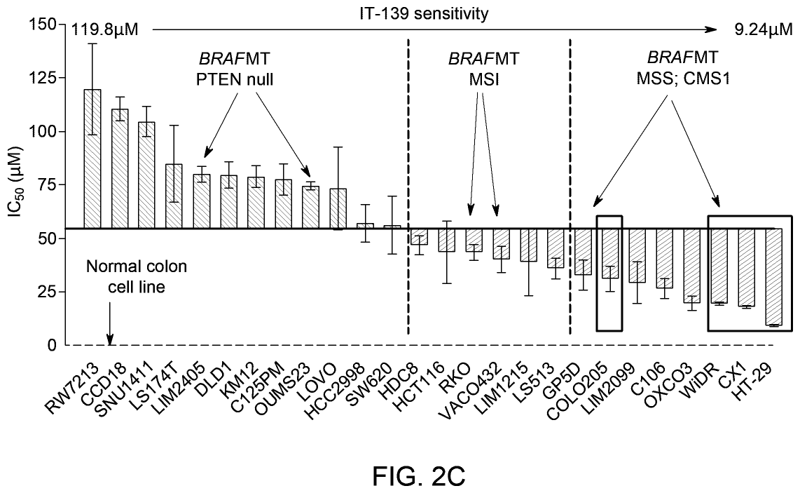

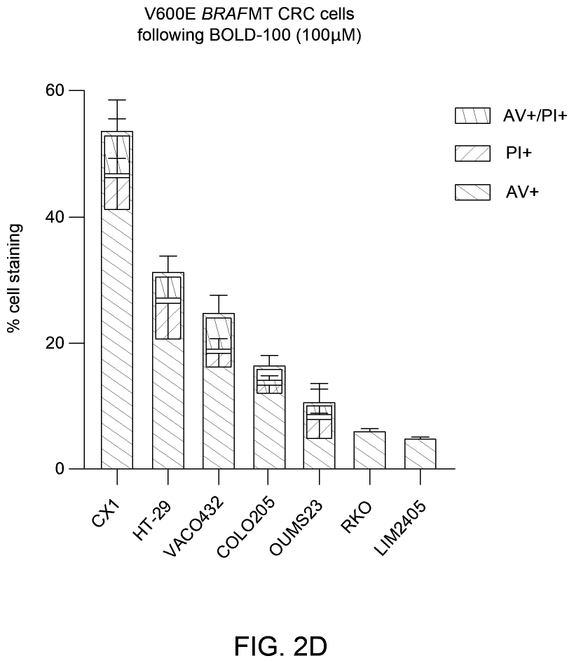

As illustrated in FIG. 2. BRAFMT, CMS1 CRC cells show increased sensitivity to treatment with BOLD-100. Upper: A. BRAFMT V600E HT-29 cells were treated with BOLD-100 for the indicated time and GRP78, CHOP and PARP levels determined by Western blotting (WB). B. HT-29 cells were treated with BOLD-100 for the indicated times and HSPA5, ATF4 and DDIT3 mRNA levels quantified using RT-PCR. Raw values were normalised to the expression of housekeeping genes ACTB and GAPDH and analysed using the ΔΔCT method. Lower: A. CRC cells were treated with increasing concentrations of BOLD-100 and cell viability determined using CellTitre-Glo® assay. IC₅₀ was calculated using Prism software package. B. CRC cells were treated with BOLD-100 for 48 h. Apoptosis was assessed using Annexin V/propidium iodide (PI) staining by high-content screening. The graph indicates the percentage of positive stained cells following treatment with 100 μM BOLD-100. Mean of 3 independent experiments is shown.

### Example 3: Oncogenic BRAF is a Determinant of Response to BOLD-100 Treatment

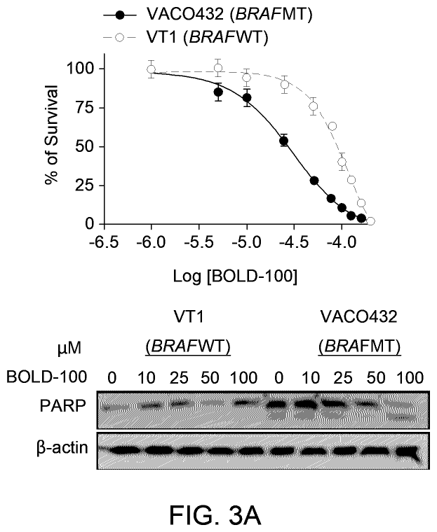

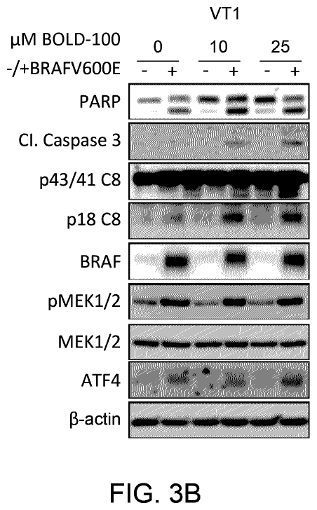

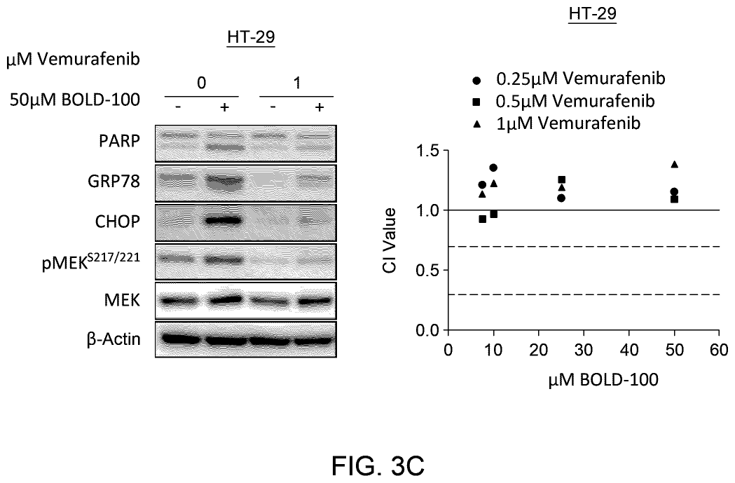

As illustrated in FIG. 3, oncogenic BRAF is a determinant of response to BOLD-100 treatment. A. Top: Isogenic BRAFMT and BRAFWT CRC cells were treated with increasing concentrations of BOLD-100 for 72 h and cell viability determined using the CellTitre-Glo® assay. IC50 was calculated using Prism software package. Lower: PARP in CRC cells treated with BOLD-100 for 48 h. B. Expression of BRAF, pMEK1/2, MEK1/2, ATF4, CHOP, PARP and cleaved C3 in VT1 CRC cells transiently transfected with 1 μg of BRAFV600E expression construct for 12 h followed by 24h treatment with BOLD-100. C. Left: CRC cells were co-treated with Vemurafenib and BOLD-100 for 48 h, and PARP, GRP78, CHOP, pMEK1/2, MEK1/2 levels determined by WB. Right: CRC cells were co-treated with no drug (control), Vemurafenib, BOLD-100 or Vemurafenib in combination with BOLD-100 for 72 h. CI values were calculated using the method of Chou and Talalay, where CI<0.3, 0.3<CI<0.7, 0.7<CI<0.85, 0.85<CI<1, CI=1, and CI>1 denotes very strong synergism, strong synergism, moderate synergism, slight synergism, an additive interaction, and antagonism, respectively.

### Example 4: BOLD-100-Induced Cell Death is Dependent on Caspase 8

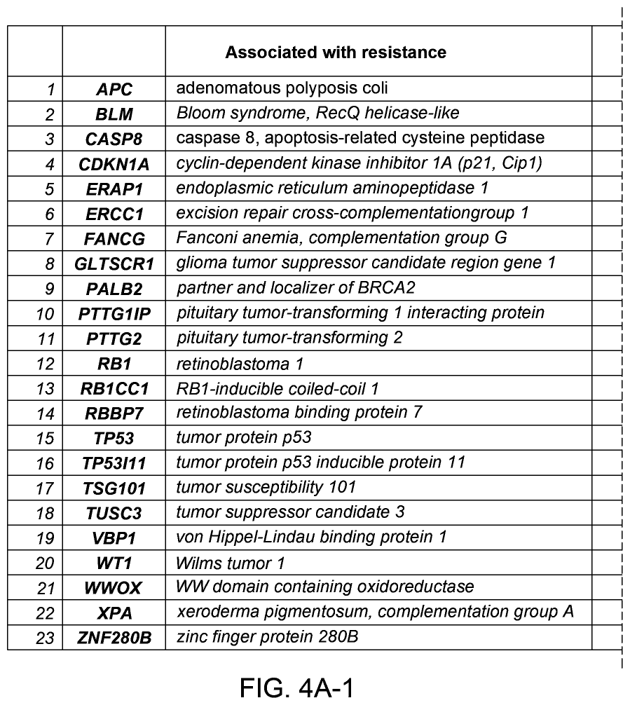

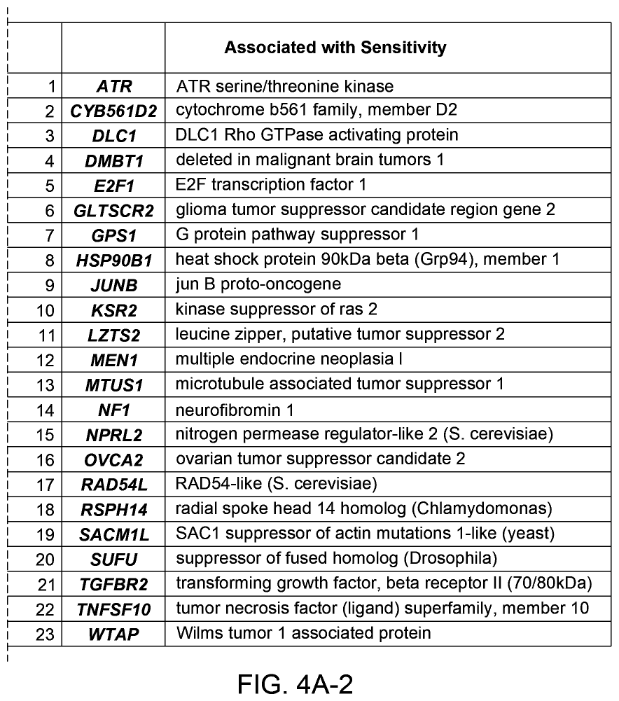

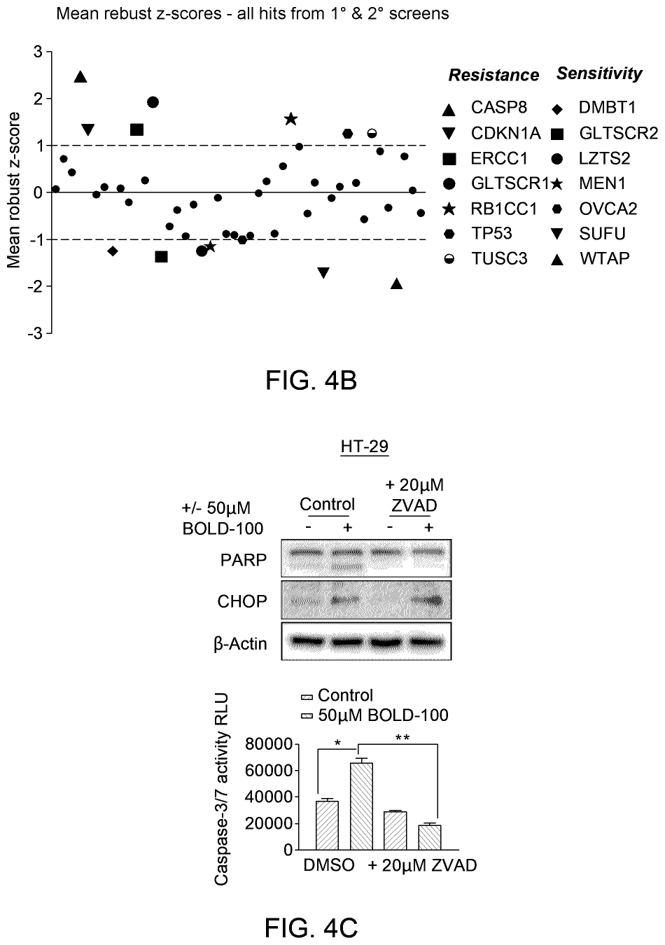

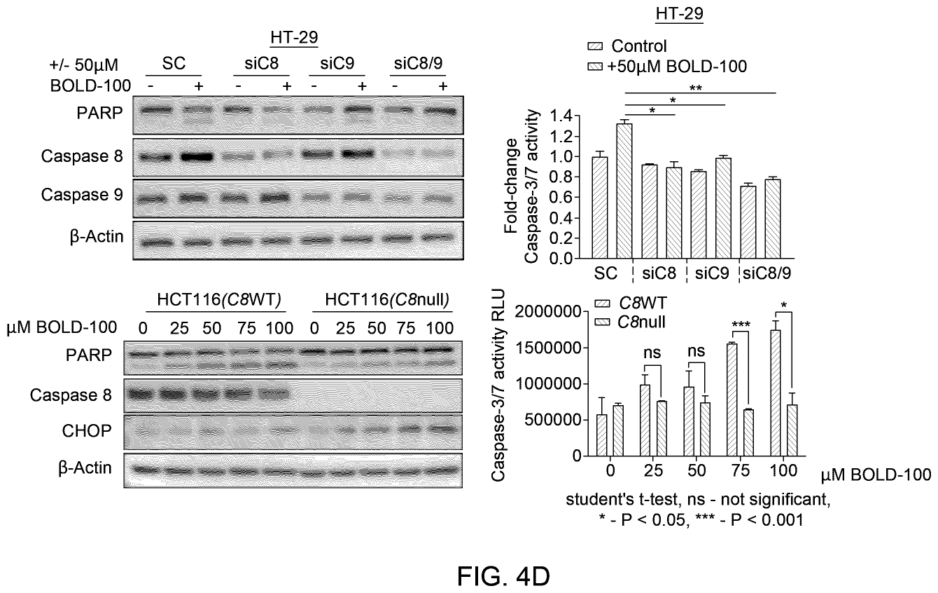

As illustrated in FIG. 4, BOLD-100 induced cell death in this model is dependent on caspase 8. A.: Positive hits for primary siRNA screen against 178 target TSG. sRNA was carried out for 24 h, followed by treatment with BOLD-100 for 48 h. Forty-Six targets were identified, resulting in either increased sensitivity or resistance to BOLD-100 (based on robust z-score ±1). B. Positive hits from secondary siRNA screen using 2 additional siRNA sequences against 46 targets. C. CRC cells were pre-incubated with DMSO or 20 μM of the pan-caspase inhibitor, z-VAD-FMK for 3 h followed by treatment with BOLD-100 for 48 h following which, apoptosis was assessed by WB analyses for PARP (top) and caspase-3/7 activity assay (bottom). D. Top: CRC cells were transfected with 10 nM C8, C9 or C8/09 siRNA for 24 h and thereafter treated with BOLD-100 for 48 h. Apoptosis was assessed by WB analysis for PARP (left) and caspase-3/7 activity (right). Bottom: Paired CRISPR HCT116 C8WT and HCT116 C8null cells were treated with BOLD-100 for 48 h. Apoptosis was determined by WB for PARP (left) and caspase-3/7 activity levels (right).

### Example 5: BOLD-100 Treatment Results in Deregulation of the DNA Damage Repair Pathway in BRAFMT CRC

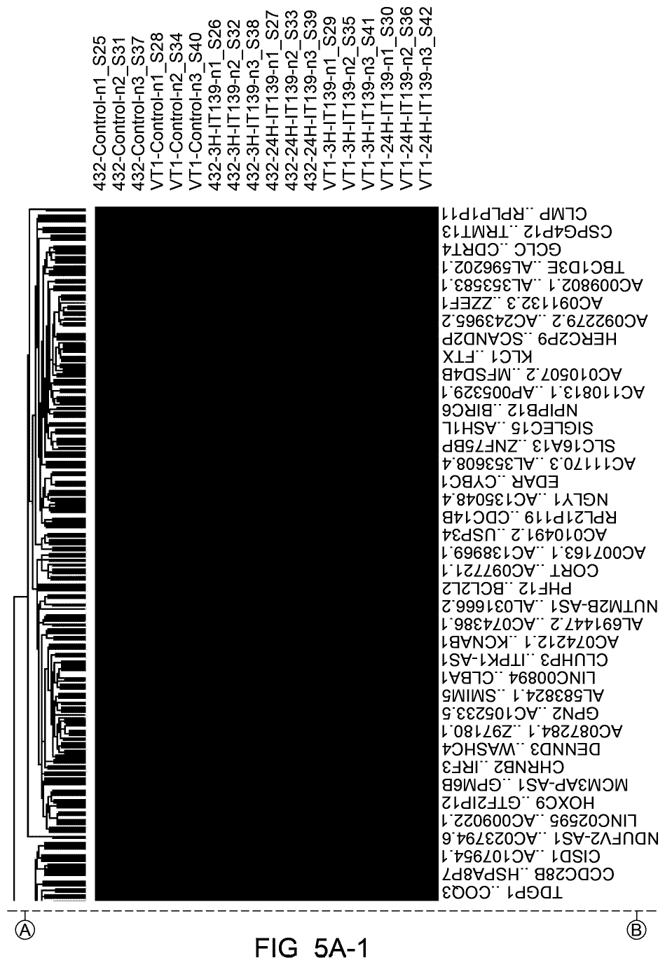

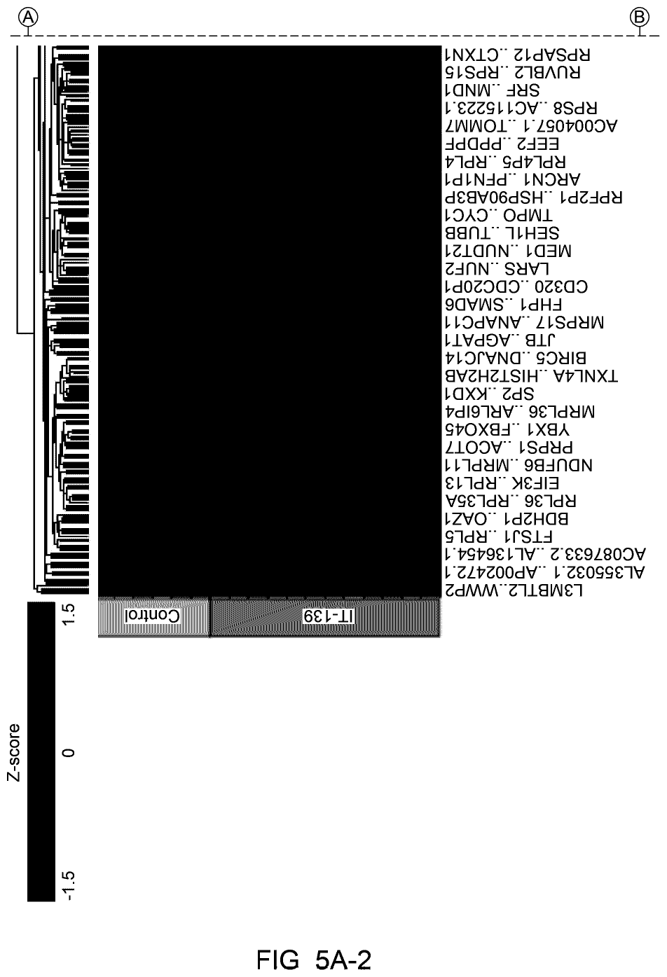

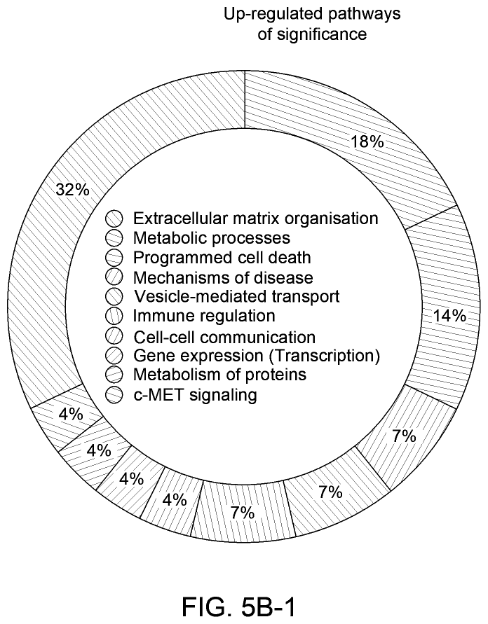

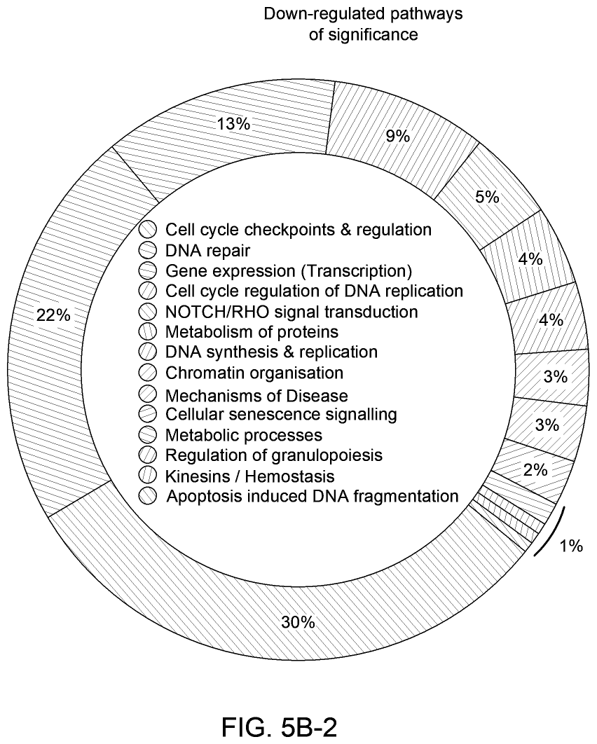

As illustrated in FIG. 5, BOLD-100 treatment results in DNA damage repair pathway activation in BRAFMT CRC: A. Heatmap of significantly (p<0.05; 1.5 fold change) downregulated and upregulated genes following 3 h and 24 h treatment with BOLD-100 in isogenic BRAFMT and WT CRC cells. B. Metacore pathway analysis of significantly down- and upregulated genes in BRAFMT VACO432 cell line.

### Example 6: ATR Inhibition Markedly Increases Response to BOLD-100 Treatment

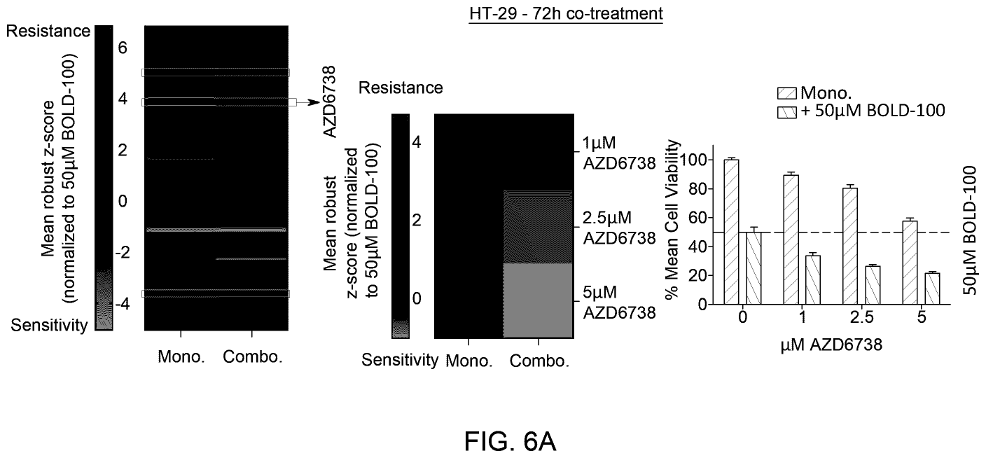

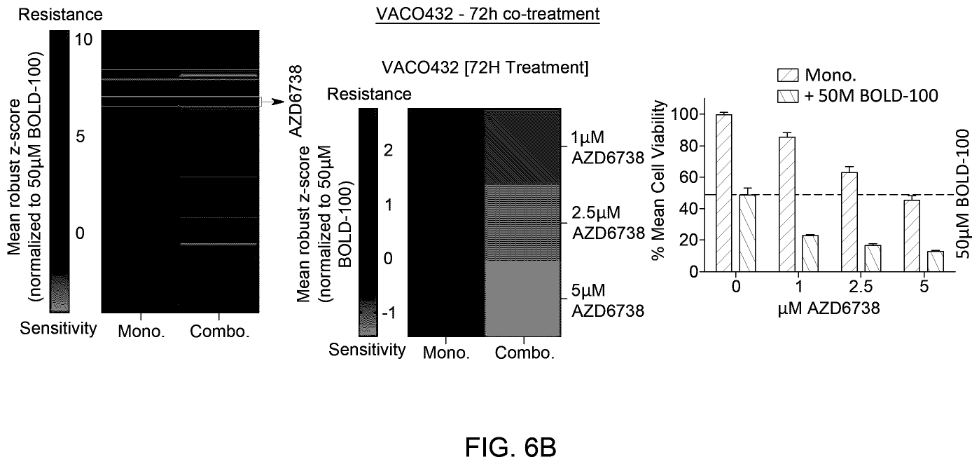

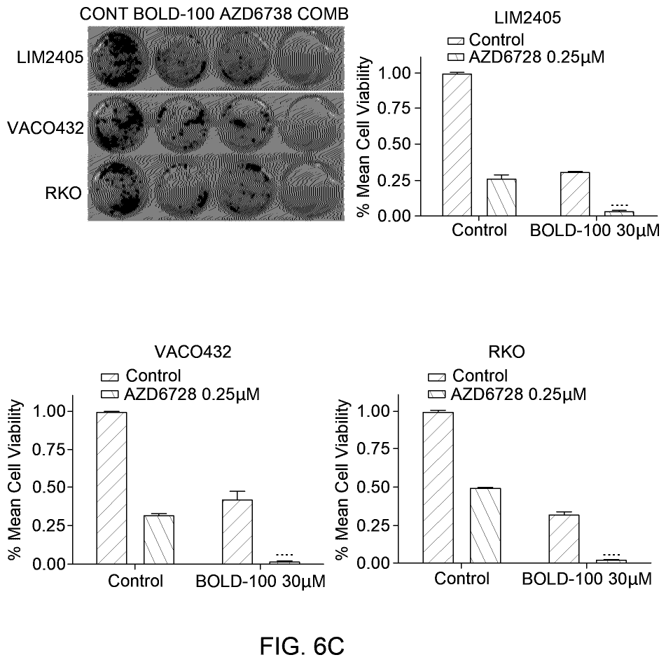

As illustrated in FIG. 6, ATR inhibition markedly increases response to BOLD-100 treatment in BRAFMT CRC cells. FDA approved drug screen targeting the significant pathways deregulated following BOLD-100 treatment in BRAFMT CRC. BRAFMT HT-29 (A) and VACO432 (B) cells were co-treated with BOLD-100, IC₁₀, IC₂₀, IC₃₀ doses of 60 FDA approved drugs alone or in combination with BOLD-100 for 72 h and cell viability determined using the CellTitre-Glo® assay. Robust z-scores were calculated for combination treatment and normalized to effect of 50 μM BOLD-100. Combinations with r-Z score of <−1 were considered to enhance sensitivity to BOLD-100 treatment. Cell viability for combined BOLD-100/AZD6738 treatment was graphed using GraphPad Prism 8.0. C. Clonogenic survival assays in BRAFMT LIM2405, VACO432 and RKO CRC cells following co-treatment with BOLD-100 and AZD6738 for 14 days. Survival was graphed using GraphPad Prism 8.0.

### Example 7: THE ATR Small Molecule Inhibitors AZD6738, M4344 and Berzosertib Increase Cell Death Following BOLD-100 Treatment in BRAFMT CRC

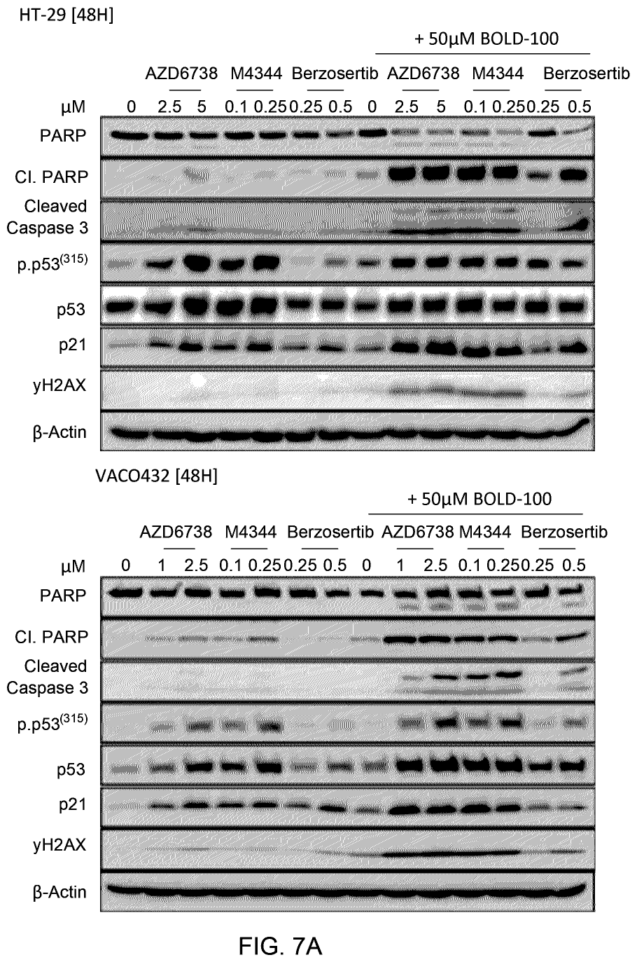

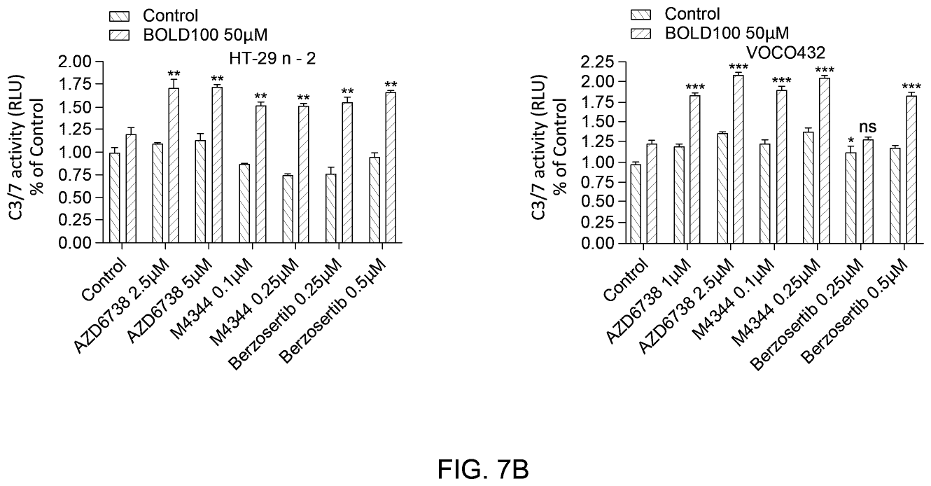

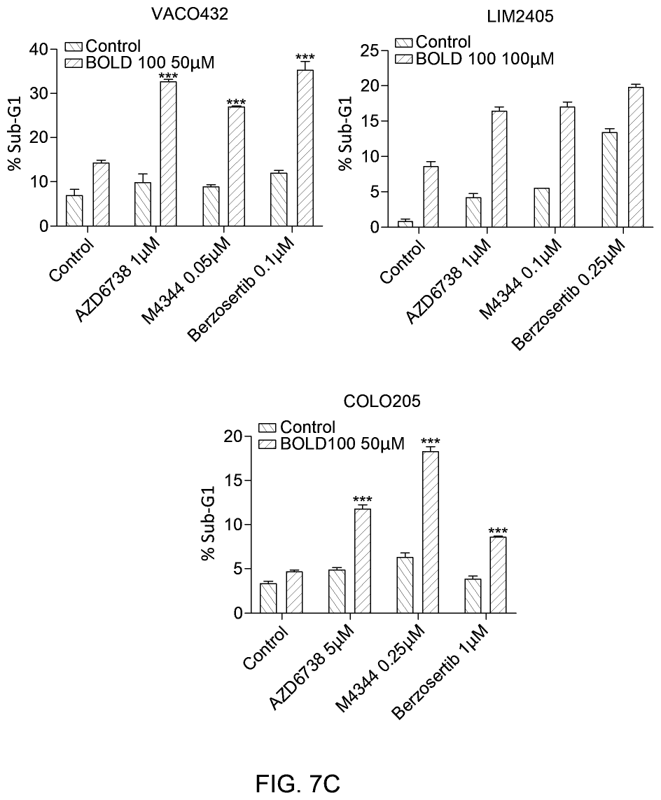

As illustrated in FIG. 7, the ATR SMI AZD6738, M4344 and Berzosertib increase cell death following BOLD-100 treatment in BRAFMT CRC. HT-29 and VACO432 CRC cells were co-treated with BOLD-100 and ATRi AZD6738, M4344 or Berzosertib for 48 h and apoptosis was assessed using WB analyses for PARP and cleaved caspase-3 (A) and caspase 3/7 activity assays (B). C. BRAFMT CRC cells VACO432, COL0205 and LIM2405 were co-treated with BOLD-100 and ATRi AZD6738, M4344 or Berzosertib for 48 h and apoptosis assessed by PI flow cytometry.

### Example 8: BOLD-100 Induces ROS-Dependent ATR/CHK1 Kinases Activation and Cell Death in BRAFMT CRC Cells

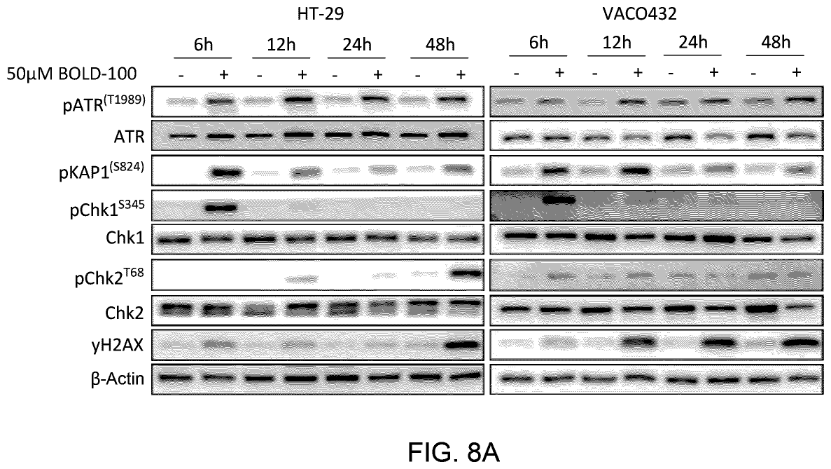

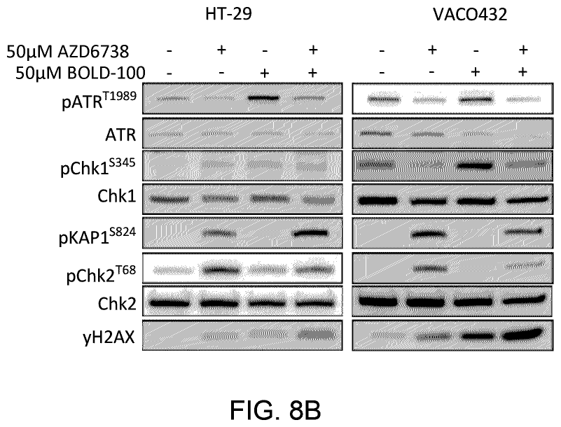

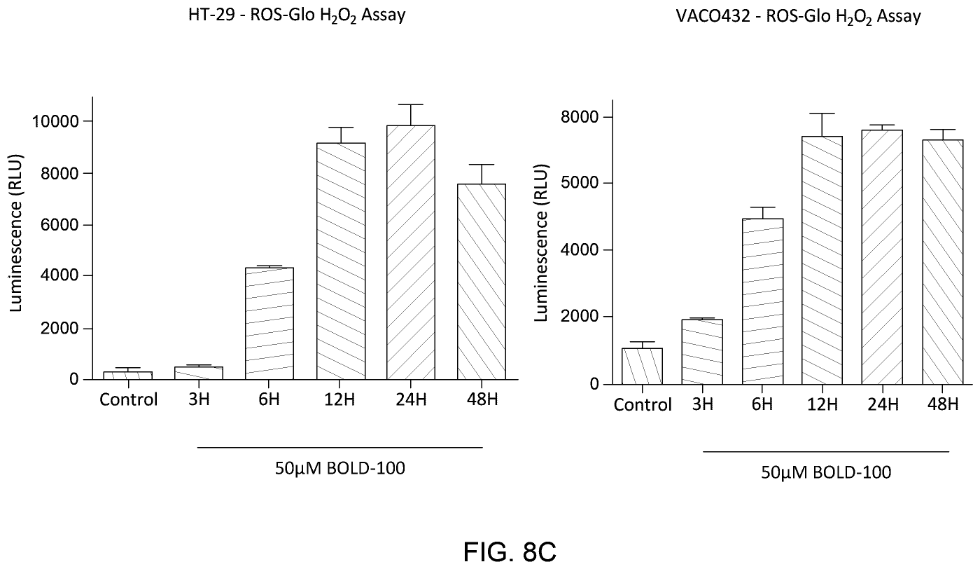

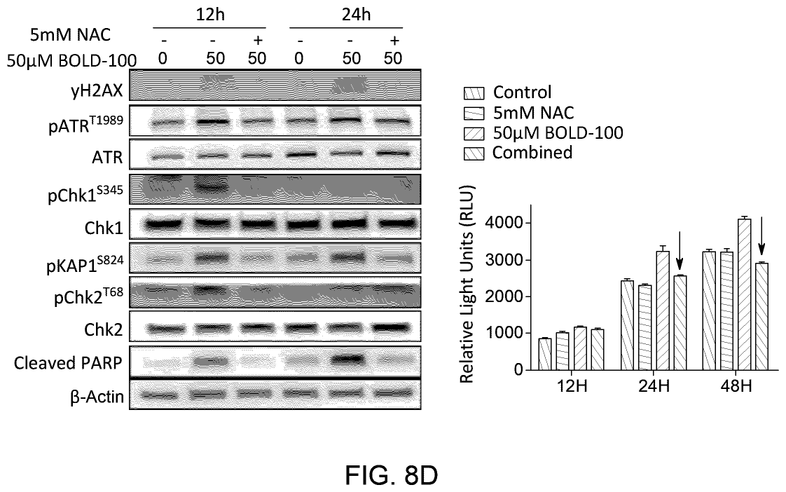

As illustrated in FIG. 8, BOLD-100 induces ROS-dependent ATR/CHK1 kinase activation and cell death in BRAFMT CRC cells. A. BRAFMT CRC cells were treated with BOLD-100 for the indicated time and ATR/KAP1/CKH1/CHK2 expression/activity measured by WB. B. CRC cells were co-treated with BOLD-100 and AZD6738 for 48 h and phosphorylation/expression of ATR/KAP1 and the downstream kinases measured. C. CRC cells were treated with BOLD-100 for the indicated times and generation of ROS measured using a ROS-Glo™ H₂O₂ assay. D. and E. WB and caspase 3/7 activity in cells pre-treated with NAC for 3 h followed by treatment with BOLD-100 for an additional 24h.

### Example 9: BOLD-100 is Most Effective in Treating Challenging CMS1 and CMS4 CRC

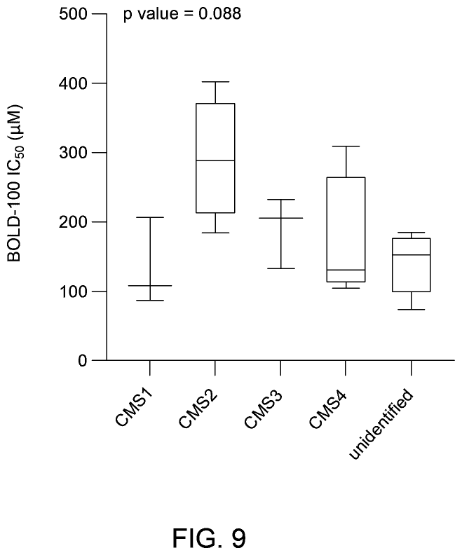

In this Example, 20 colon cancer cell lines were treated with increasing concentrations of BOLD-100 for 72 hours and viability was measured by the standard Cell Titer-Glo (CTG) luminescent cell viability assay. The IC₅₀ of each cell line was determined from the dose response curves using the Prism software package. The colon cancer cell lines were classified into different subtypes based on the Consensus Molecular Subtype (CMS) classification of CRC preclinical models. As illustrated in FIG. 9, the CMS1 and CMS4 subtypes were shown to be most responsive to BOLD-100 monotherapy treatment. The distribution of minimum to maximum IC₅₀ is indicated by the 2-way bars and the median is indicated by the horizontal bar. The CMS1 subtype, which harbors BRAF mutations, and the CMS4 subtypes have the worst overall survival and response rates, demonstrating that BOLD-100 is surprisingly effective in the treatment of colon cancer in the difficult to treat subtypes.

### Example 10: ATR Inhibition Markedly Increases Response to BOLD-100 Treatment in Multiple Myeloma

Myeloma is a lymphoid malignancy involving plasma cells primarily resident in the bone marrow, although malignant plasma cells may also be seen in peripheral blood, soft tissue and organs. Myeloma is accordingly a plasma cell dyscrasia, which manifests in forms that may be distinguished by the affected sites, in multiple myeloma several different areas are affected, in plasmacytoma only one site is affected, in localized myeloma adjacent sites are affected, and in extramedullary myeloma there is involvement of tissue other than bone marrow. As used herein, the term “myeloma” accordingly refers to the spectrum of diseases recognized in the art as such. Within this spectrum of disease, relapsed MM is generally regarded as a recurrence of the disease after prior response, typically based on objective clinical criteria, and relapsed/refractory MM (RRMM) is generally defined as a disease which becomes non-responsive or progressive on therapy or within 60 days of the last treatment in patients who had achieved a minimal response (MR) or better on prior therapy. The treatment of RRMM poses particular challenges.

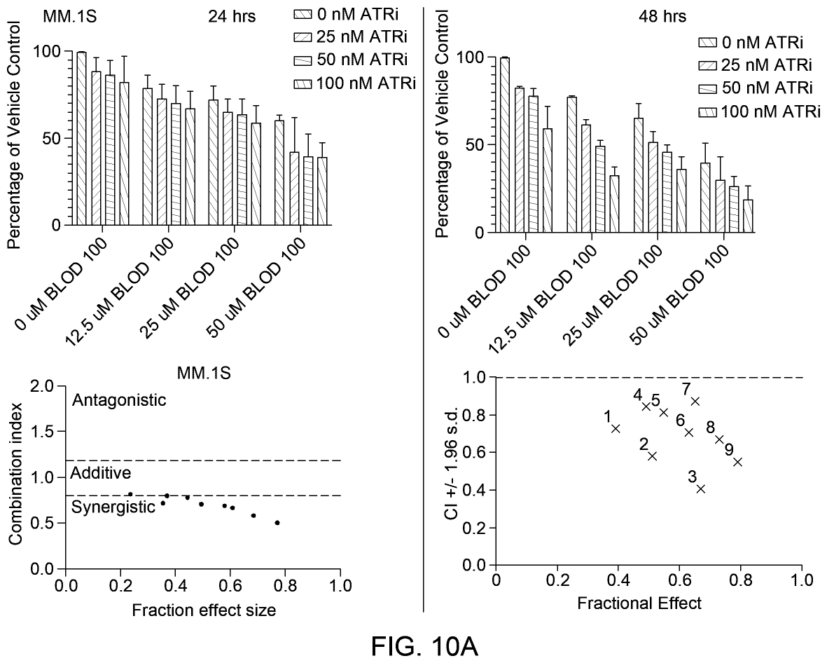

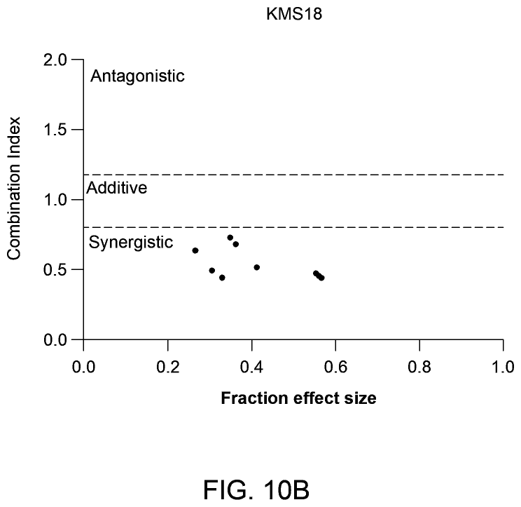

In this Example, multiple myeloma cell lines were treated with increasing doses of BOLD-100 in combination with increasing doses of the ATR inhibitor BAY 1895344 for either 24 (FIG. 10 (A) left panels) or 48 hours (FIG. 10 (A) right panels), and the cell viability was measured by standard Cell Titer-Glo (CTG) luminescent cell viability assay. FIG. 10 (A) shows the response in the multiple myeloma cell line MM.1S while FIG. 10 (B) shows the response in the multiple myeloma cell line KMS18. Bar graphs represent the number of cells alive in the assay with the MM.1S cells, as compared to the vehicle controls. The scatter plots represent the degree of synergy between the drug combinations at different dose levels. In the scatter plots, compound interactions were calculated as a Combination Index (CI) by multiple drug effect analysis, performed by the median equation principle according to the methodology described by Chou and Talalay using the Compusyn software, version 1.0 (see Chou TC. “Drug combination studies and their synergy quantification using the Chou-Talalay method.” Cancer Res. 2010 Jan. 15; 70(2):440-6). The CI values are one way to indicate synergistic, additive or antagonistic behavior of the drug combination. Using this method, Oki, =1, and >1 indicate synergism, additive effect and antagonism, respectively. Synergistic interactions of the two drugs to reduce cell viability was observed.

### REFERENCES

Venderbosch S, Nagtegaal I D, Maughan T S, et al. Clin Cancer Res. 2014; 20(20):5322-30.
Lochhead P, Kuchiba A, Imamura Y, et al. J Natl Cancer Inst. 2013; 105(15):1151-6.
Kopetz, S. et al. Encorafenib, Binimetinib, and Cetuximab in BRAF V600E-Mutated Colorectal Cancer. N Engl J Med. 381, 1632-1643 (2019).
Guinney, J. et al. The consensus molecular subtypes of colorectal cancer. Nat Med. 21, 1350-1356 (2015).
Medico, E. et al. The molecular landscape of colorectal cancer cell lines unveils clinically actionable kinase targets. Nature communications. 6, 7002 (2015).
Burris, H. A. et al. Safety and activity of IT-139, a ruthenium-based compound, in patients with advanced solid tumours: a first-in-human, open-label, dose-escalation phase I study with expansion cohort. ESMO open. 1, e000154 (2016).

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Terms such as “exemplary” or “exemplified” are used herein to mean “serving as an example, instance, or illustration.” Any implementation described herein as “exemplary” or “exemplified” is accordingly not to be construed as necessarily preferred or advantageous over other implementations, all such implementations being independent embodiments. Unless otherwise stated, numeric ranges are inclusive of the numbers defining the range, and numbers are necessarily approximations to the given decimal. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification, and all documents cited in such documents and publications, are hereby incorporated herein by reference as if each individual publication were specifically and individually indicated to be incorporated by reference herein and as though fully set forth herein. The invention includes all embodiments and variations substantially as hereinbefore described and with reference to the examples and drawings.

In some embodiments, the invention excludes steps that involve medical or surgical treatment.

1. A method for treating a cancer in a human patient in need thereof, comprising administering an effective amount of sodium trans-[tetrachloridobis(1H-indazole)ruthenate(III)] and an effective amount of an inhibitor of ataxia-telangiectasia mutated and Rad3-related protein kinase (ATRi).

2. The method of claim 1, wherein the effective amount of sodium trans-[tetrachloridobis(1H-indazole)ruthenate(III)] and the effective amount of the ATRi are synergistically effective for treating the cancer.

3. The method of claim 1, wherein the cancer is a cancer that is resistant to treatment with sodium trans-[tetrachloridobis(1H-indazole)ruthenate(III)] alone, or is a cancer that is resistant to treatment with the ATRi alone, or a cancer that is resistant to another chemotherapy agent or chemotherapy regimen.

4. The method according to claim 1, wherein the cancer is a colorectal cancer (CRC) or a myeloma.

5. The method of claim 4, wherein the CRC is an adenocarcinoma, or the myeloma is a multiple myeloma.

6. The method according to claim 1, wherein the cancer is characterized by a BRAF mutation (BRAFMT).

7. The method according to claim 1, wherein the cancer is characterized by microsatellite stability (MSS).

8. A method for treating a cancer in a human patient in need thereof, comprising administering an effective amount of sodium trans-[tetrachloridobis(1H-indazole)ruthenate(III)], wherein the cancer is a colorectal cancer (CRC) characterized by a BRAF mutation (BRAFMT) or a CRC of consensus molecular subtype (CMS) CMS1 or CMS4.

9. The method of claim 8, wherein the BRAFMT CRC is an adenocarcinoma.

10. The method of claim 8, wherein the BRAFMT CRC is characterized by microsatellite stability (MSS).

11. The method of claim 8, wherein the sodium trans-[tetrachloridobis(1H-indazole)ruthenate(III)] is administered in combination with an effective amount of an inhibitor of ataxia-telangiectasia mutated and Rad3-related protein kinase (ATRi).

12. The method of claim 11, wherein the effective amount of sodium trans-[tetrachloridobis(1H-indazole)ruthenate(III)] and the effective amount of ATRi are synergistically effective for treating the cancer.

13. The method of claim 11 or 12, wherein the cancer is a cancer that is resistant to treatment with sodium trans-[tetrachloridobis(1H-indazole)ruthenate(III)] alone, or is a cancer that is resistant to treatment with the ATRi alone, or a cancer that is resistant to another chemotherapy agent or chemotherapy regimen.

14. The method according to claim 1, wherein the sodium trans-[tetrachloridobis(1H-indazole)ruthenate(III)] and the ATRi are administered sequentially, in any order.

15. The method according to claim 1, wherein the sodium trans-[tetrachloridobis(1H-indazole)ruthenate(III)] and the ATRi are administered in combination, in a co-formulation or separately.

16. The method of claim 1, further comprising administering an inhibitor of expression one or more of the following genes: ATR (ATR serine/threonine kinase; CYB561D2 (cytochrome b561 family, member D2); DLC1 (DLC1 Rho GTPase activating protein); DMBT1 (deleted in malignant brain tumors 1); E2F1 (E2F transcription factor 1); GLTSCR2 (glioma tumor suppressor candidate region gene 2); GPS1 (G protein pathway suppressor 1); HSP90B1 (heat shock protein 90 kDa beta (Grp94), member 1); JUNB (jun B proto-oncogene); KSR2 (kinase suppressor of ras 2); LZTS2 (leucine zipper, putative tumor suppressor 2); MEN1 (multiple endocrine neoplasia I); MTUS1 (microtubule associated tumor suppressor 1); NF1 (neurofibromin 1); NPRL2 (nitrogen permease regulator-like (S. cerevisiae)); OVCA2 (ovarian tumor suppressor candidate 2); RAD54L (RAD54-like (S. cerevisiae)); RSPH14 (radial spoke head 14 homolog (Chlamydomonas)); SACM1L (SAC1 suppressor of actin mutations 1-like (yeast)); SUFU (suppressor of fused homolog (Drosophila)); TGFBR2 (transforming growth factor, beta receptor II (70/80 kDa)); TNF SF10 (tumor necrosis factor (ligand) superfamily, member 10); WTAP (Wilms tumor 1 associated protein).

17. The method of claim 16 wherein the inhibitor of expression is an siRNA.

18. The method of claim 1, further comprising assaying a sample from the patient for a mutation in one or more of the following genes: ATR (ATR serine/threonine kinase; CYB561D2 (cytochrome b561 family, member D2); DLC1 (DLC1 Rho GTPase activating protein); DMBT1 (deleted in malignant brain tumors 1); E2F1 (E2F transcription factor 1); GLTSCR2 (glioma tumor suppressor candidate region gene 2); GPS1 (G protein pathway suppressor 1); HSP90B1 (heat shock protein 90 kDa beta (Grp94), member 1); JUNB (jun B proto-oncogene); KSR2 (kinase suppressor of ras 2); LZTS2 (leucine zipper, putative tumor suppressor 2); MEN1 (multiple endocrine neoplasia I); MTUS1 (microtubule associated tumor suppressor 1); NF1 (neurofibromin 1); NPRL2 (nitrogen permease regulator-like 2 (S. cerevisiae)); OVCA2 (ovarian tumor suppressor candidate 2); RAD54L (RAD54-like (S. cerevisiae)); RSPH14 (radial spoke head 14 homolog (Chlamydomonas)); SACM1L (SAC1 suppressor of actin mutations 1-like (yeast)); SUFU (suppressor of fused homolog (Drosophila)); TGFBR2 (transforming growth factor, beta receptor II (70/80 kDa)); TNF SF10 (tumor necrosis factor (ligand) superfamily, member 10); WTAP (Wilms tumor 1 associated protein); APC (adenomatous polyposis coli); BLM (Bloom syndrome, RecQ helicase-like); CASP8 (caspase 8, apoptosis-related cysteine peptidase); CDKN1A (cyclin-dependent kinase inhibitor 1A (p21, Cip1)); ERAP1 (endoplasmic reticulum aminopeptidase 1); ERCC1 (excision repair cross-complementation group 1); FANCG (Fanconi anemia, complementation group G); GLTSCR1 (glioma tumor suppressor candidate region gene 1); PALB2 (partner and localizer of BRCA2); PTTG1IP (pituitary tumor-transforming 1 interacting protein); PTTG2 (pituitary tumor-transforming 2); RB1 (retinoblastoma 1); RB1CC1 (RB1-inducible coiled-coil 1); RBBP7 (retinoblastoma binding protein 7); TP53 (tumor protein p53); TP53I11 (tumor protein p53 inducible protein 11); TSG101 (tumor susceptibility 101); TUSC3 (tumor suppressor candidate 3); VBP1 (von Hippel-Lindau binding protein 1); WT1 (Wilms tumor 1); WWOX (WW domain containing oxidoreductase); XPA (xeroderma pigmentosum, complementation group A); and/or ZNF280B (zinc finger protein 280B).
